(19) United States
(12) Patent Application Publication
(10) Pub. No.: US 2022/0127251 A1
(43) Pub. Date: Apr. 28, 2022

(54) PROCESS FOR THE PRODUCTION OF ARYLAMINES

(71) Applicant: Merck Patent GmbH, Darmstadt (DE)

(72) Inventors: Christian EHRENREICH, Darmstadt (DE); Dominik JOOSTEN, Ober-Ramstadt (DE); Philipp Hans FACKLER, Frankfurt Am Main (DE); Thorsten VOM STEIN, Darmstadt (DE); Stefan LEHMANN, Otzberg (DE); Caroline WERN, Ottweiler (DE)

(21) Appl. No.: 17/257,738

(22) PCT Filed: Jul. 1, 2019
(86) PCT No.: PCT/EP2019/067535

§ 371 Date: Jan. 4, 2021

(30) Foreign Application Priority Data
Jul. 4, 2018 (EP) 18181706.5

(51) Int. Cl. C07D 405/04; C07C 217/84; C07D 209/86; C07C 209/68

(52) U.S. Cl. CPC C07D 405/04; C07C 209/68; C07D 209/86; C07C 217/84

(57) ABSTRACT

The invention relates to a process for producing compounds containing at least one arylamino group by means of a palladium-catalysed coupling reaction of an arylamino compound with an aryl compound, using LiOtBu as a base.

The invention relates to a process for preparing compounds containing at least one arylamino group by palladium-catalyzed coupling reaction of an amino compound with an aryl compound.

The forming of a bond between a nitrogen atom and an aryl or heteroaryl group is a key reaction in organic synthesis. Accordingly, aryl- and heteroarylamino compounds are often important intermediates in multistage syntheses. Moreover, aryl- and heteroarylamino compounds find use as active pharmaceutical ingredients, and as functional materials, for example in electronic devices. In all cases, the achievement of a high yield in the coupling step and the avoidance of by-products are of major significance, since syntheses on the industrial scale are otherwise achievable only with difficulty. Furthermore, it is indispensable for achievement of a high product purity that side reactions are very substantially avoided. Also desirable is a high selectivity in the presence of further functional groups.

The prior art discloses processes for synthesis of arylamino compounds in which an amino compound and an aryl compound as reactants are reacted under palladium catalysis in the presence of a base (Hartwig-Buchwald coupling). Such processes are described inter alia in U.S. Pat. No. 5,576,460, in Guram et al. (Angew. Chem., Int. Ed. 1995, 43, 1348), in Louie et al. (Tetrahedron Lett. 1995, 36, 3609), and in Surry et al. (Chemical Science 2011, 2, 27).

Attempts known in the art to further develop the method relate predominantly to the use of novel ligand catalyst systems (Surry et al., Chemical Science 2011, 2, 27). According to the prior art, typically NaOtBu is used in the Hartwig-Buchwald reaction (see literature references cited above or Kuwano et al., Synlett 2010, 1819). Additionally known is the use of inorganic bases such as KOH, NaOH, Cs₂CO₃ or K₃PO₄ (see literature references cited above), as is the use of very strong bases, such as alkali metal amides or alkyllithium (WO 2013/068075).

In spite of the good efficiency of these processes overall and their breadth of applicability, there is a need for an improvement in the method, especially with regard to slow-reacting and/or sterically demanding reactants, and with regard to hydrolysis-sensitive reactants, for example those with triflates or other sulfonates as leaving group. There is still a need for an improvement in the process with regard to the product yield and reduction in the formation of by-products, especially formation of the defunctionalized aryl compound. The problems mentioned occur especially when sterically hindered reactants are used, for example ortho-substituted aryl compounds or secondary amino compounds having sterically demanding substituents. The formation of the hydrolyzed aryl compound as by-product occurs especially in the case of use of triflate or other sulfonates as leaving group. Also desirable is an improvement in selectivity in the presence of multiple different groups that can serve as leaving group.

It has been found that, surprisingly, lithium tert-butoxide (LiOtBu) is of excellent suitability as base for use in palladium-catalyzed C—N coupling reactions (Hartwig-Buchwald coupling) for the coupling of aryl- or heteroarylamines. This is especially true when sulfonates are used as leaving group. Compared to the use of NaOtBu, which is typically used in C—N coupling reactions, or KOtBu, it is thus possible to achieve distinctly improved yields. At the same time, when sulfonates are used as leaving group, a much lower level of side reactions is observed, especially a much lower level of hydrolysis of the sulfonate leaving group. Furthermore, the use of LiOtBu as base enables selective reaction of sulfonate groups, especially of triflate, in the presence of chlorine substituents. No such selectivity is observed with NaOtBu as base.

The use of LiOtBu as base for the coupling of aliphatic amines is already described by Louie et al. (Tetrahedron Lett. 1995, 36, 3609). However, this reaction with aliphatic amines gives virtually no conversion.

The invention therefore provides a process for preparing a secondary or tertiary arylamino compound by a palladium-catalyzed coupling reaction between a primary or secondary arylamino compound and an aryl compound, characterized in that the process is conducted in the presence of lithium tert-butoxide (LiOtBu) as base.

The use of a primary arylamino compound, according to the catalyst, leads to a secondary or tertiary arylamino compound, and the use of a secondary arylamino compound leads to a tertiary arylamino compound, as reaction product. In a preferred embodiment of the invention, a secondary arylamino compound is used in the process of the invention, and the product is a tertiary arylamino compound.

In the context of the present invention, an arylamino compound is understood to mean an amine to which at least one optionally substituted aromatic or heteroaromatic ring system is bonded. A primary arylamino compound here is a compound containing a primary amino group —NH₂ bonded to an optionally substituted aromatic or heteroaromatic ring system. A secondary arylamino compound is a compound containing a secondary amino group —NH— bonded to two optionally substituted aromatic or heteroaromatic ring systems. It is also possible here for one aromatic ring system and one heteroaromatic ring system to be bonded to the nitrogen. A tertiary arylamino compound is a compound containing a tertiary nitrogen atom N bonded to three optionally substituted aromatic or heteroaromatic ring systems. It is also possible here for one aromatic ring system and two heteroaromatic ring systems or two aromatic ring systems and one heteroaromatic ring system to be bonded to the nitrogen. It is possible here for the aromatic or heteroaromatic ring systems to be the same or different. In addition, the aromatic or heteroaromatic ring systems in the secondary or tertiary amine may also be joined to one another to form a ring via a single bond or a bivalent group. For example, the reaction of a carbazole is also possible.

In the context of the present invention, an aryl compound is understood to mean an optionally substituted organic compound containing at least one aromatic or heteroaromatic ring system. The aryl compound here contains a leaving group which is eliminated in the coupling reaction of the invention. Preferred leaving groups are listed below.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused aryl or heteroaryl group, for example naphthalene, anthracene, phenanthrene, quinoline, isoquinoline, etc.

An aromatic ring system in the context of this invention contains 6 to 60 carbon atoms in the ring system, preferably 6 to 40 carbon atoms. A heteroaromatic ring system in the context of this invention contains 1 to 60 carbon atoms, preferably 1 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ether, stilbene, etc. shall also be regarded as aromatic ring systems in the context of this invention. In addition, systems in which two or more aryl or heteroaryl groups are bonded directly to one another, for example biphenyl, terphenyl, quaterphenyl or bipyridine, shall likewise be regarded as an aromatic or heteroaromatic ring system.

In the context of the present invention, a C₁- to C₂₀-alkyl group in which individual hydrogen atoms or CH₂ groups may also be substituted by the abovementioned groups is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals. An alkenyl group is understood to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is understood to mean, for example, ethynyl, propynyl, butynyl, pentynyl, hexynyl, heptynyl or octynyl. A C₁- to C₄₀-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy. A cyclic alkyl, alkoxy or thioalkoxy group in the context of this invention is understood to mean a monocyclic, bicyclic or polycyclic group.

An aromatic or heteroaromatic ring system which has 5-60 aromatic ring atoms and may also be substituted in each case by the abovementioned radicals and which may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, cis- or trans-monobenzoindenofluorene, cis- or trans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, thiazine, oxazine, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, isothiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, isoxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine, coumarin and benzothiadiazole.

The wording that two or more radicals or groups together may form a ring, in the context of the present description, should be understood to mean, inter alia, that the two radicals or groups are joined to one another by a chemical bond with formal elimination of two hydrogen atoms. This is illustrated by the following scheme:

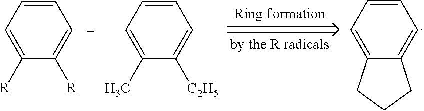

In addition, however, the abovementioned wording shall also be understood to mean that, if one of the two radicals is hydrogen, the second radical binds to the position to which the hydrogen atom was bonded, forming a ring. This shall be illustrated by the following scheme:

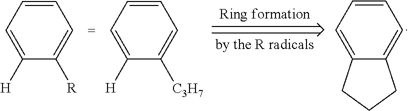

A palladium-catalyzed coupling reaction in the context of the present invention is understood to mean a reaction between two organic compounds in which a single bond is formed between the two compounds under palladium catalysis. This is effected with formal elimination of a small molecule. It is preferable that no further reactions of the two compounds occur here. According to the invention, the coupling reaction is effected between an arylamino compound and an aryl compound, where the aryl compound has a leaving group preferably selected from the group consisting of optionally substituted alkylsulfonate, optionally substituted arylsulfonate, halide and diazonium. In the coupling reaction, the single bond formed replaces the N—H bond in the arylamino compound and the bond to the leaving group in the aryl compound.

A preferred embodiment of the process of the invention corresponds to the following scheme:

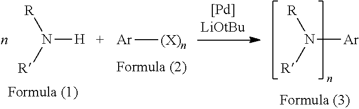

where the symbols and indices used are as follows:

Ar is an aromatic or heteroaromatic ring system which has 5 to 60 aromatic ring atoms and may be substituted by one or more R¹ radicals;

R is the same or different at each instance and is an aromatic or heteroaromatic ring system which has 5 to 60 aromatic ring atoms and may be substituted in each case by one or more R¹ radicals;

R′ is the same or different and is R or is H or D; at the same time, it is possible for R and R′ to be joined to one another directly or via an R¹ group and hence form a ring system together with the nitrogen atom to which they bind;

R¹ is the same or different at each instance and is selected from H, D, F, Cl, Br, I, B(OR²)₂, CHO, C(═O)R², CR²═C(R²)₂, CN, C(═O)OR², C(═O)N(R²)₂, Si(R²)₃, N(R²)₂, NO₂, P(═O)(R²)₂, OSO₂R², OR², S(═O)R², S(═O)₂R², a straight-chain alkyl, alkoxy or thioalkyl group having 1 to 20 carbon atoms or a branched or cyclic alkyl, alkoxy or thioalkyl group having 3 to 20 carbon atoms or an alkenyl or alkynyl group having 2 to 20 carbon atoms, where the abovementioned groups may each be substituted by one or more R² radicals and where one or more CH₂ groups in the abovementioned groups may be replaced by —R²C═CR²—, —C═C—, Si(R²)₂, C═O, C═S, C═NR², —C(═O)O—, —C(═O)NR²—, NR², P(═O)(R²), —O—, —S—, SO or SO₂ and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, or an aryl- or heteroaryloxy group which has 5 to 30 aromatic ring atoms and may be substituted by one or more R² radicals; at the same time, it is possible for two or more R¹ radicals to be joined to one another and hence form a ring;

R² is the same or different at each instance and is selected from H, D, F, Cl, Br, I, B(OR³)₂, CHO, C(═O)R³, CR³═C(R³)₂, CN, C(═O)OR³, C(═O)N(R³)₂, Si(R³)₃, N(R³)₂, NO₂, P(═O)(R³)₂, OSO₂R³, OR³, S(═O)R³, S(═O)₂R³, a straight-chain alkyl, alkoxy or thioalkyl group having 1 to 20 carbon atoms or a branched or cyclic alkyl, alkoxy or thioalkyl group having 3 to 20 carbon atoms or an alkenyl or alkynyl group having 2 to 20 carbon atoms, where the abovementioned groups may each be substituted by one or more R³ radicals and where one or more CH₂ groups in the abovementioned groups may be replaced by —R³C═CR³—, —C≡C—, Si(R³)₂, C═O, C═S, C═NR³, —C(═O)O—, —C(═O)NR³—, NR³, P(═O)(R³), —O—, —S—, SO or SO₂ and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R³ radicals, or an aryl- or heteroaryloxy group which has 5 to 30 aromatic ring atoms and may be substituted by one or more R³ radicals; at the same time, it is possible for two or more R² radicals to be joined to one another and hence form a ring;

R³ is the same or different at each instance and is H, D, F or an aliphatic, aromatic or heteroaromatic organic radical having 1 to 20 carbon atoms, in which one or more hydrogen atoms may also be replaced by D or F; at the same time, two or more R³ substituents may be joined to one another and hence may form a ring;

X is the same or different at each instance and is a leaving group;

n is an integer from 1 to 10.

The compound of the formula (1) here

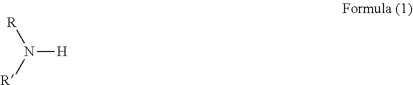

is the abovementioned primary (when R′ ═H or D) or secondary (when R′ is the same or different and is R) arylamino compound.

The compound of the formula (2), Ar—(X)ₙ, is the abovementioned aryl compound, where X represents the leaving group.

The compound of the formula (3)

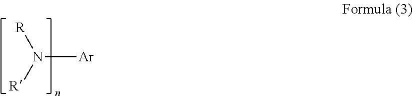

is the abovementioned secondary (when R′═H or D) or tertiary (when R′ is the same or different and is R) arylamino compound which is the reaction product.

In addition, in the reaction equation shown above, [Pd] represents the palladium compound which is used as catalyst.

In a preferred embodiment, R′ is the same or different and is R, and so preference is given to using a secondary arylamino compound for preparation of a tertiary arylamino compound.

The process of the invention is also suitable for synthesis of oligomeric or polymeric arylamino compounds. Such a reaction can be effected by reaction of an aryl compound having at least two leaving groups with an arylamino compound having at least two amino groups. The basic reaction scheme shown above, comprising bond formation between a nitrogen atom and an aryl group, is still applicable in this regard.

Preference is given to using the process of the invention for preparation of small organic compounds, i.e. compounds having a molecular weight of less than 5000 Da, more preferably less than 3000 Da and most preferably less than 2000 Da.

Preferred arylamino compounds as reactants are diarylamino compounds. Suitable arylamino compounds are especially also those arylamino compounds in which one or both of the R and R′ groups bonded to the nitrogen atom are sterically demanding. It is also possible here for one or both R and/or R′ groups to constitute an aromatic or heteroaromatic ring system bearing a substituent in the ortho position to the bond to the nitrogen atom, or an aromatic or heteroaromatic ring system bearing a fused-on ring in ortho position to the bond to the nitrogen atom. Specifically also with arylamino compounds bearing sterically demanding R or R′ groups, the process of the invention leads to very good yields, whereas the yields for such amines with NaOtBu as base are much poorer or, according to the substrate, no conversion at all is observed.

What is meant by the expression “sterically demanding” in the context of the present invention is that a group or substituent has a large spatial extent. The presence of a sterically demanding group typically leads to slowing of reactions in positions adjacent to or in spatial proximity to this group. In the extreme case, the reaction is slowed to such a degree that it is no longer preparatively utilizable. Steric hindrance can be caused by any groups and increases with the spatial extent (steric demand) of the group. The steric demand rises, for example, in the order of H, methyl, ethyl, isopropyl, tert-butyl, such that H is the least sterically demanding group and tert-butyl the most sterically demanding group in this series.

Suitable aromatic or heteroaromatic ring systems in the arylamino compound, or suitable R and R′ groups in formula (1) if R′ is not H or D, are preferably the same or different at each instance and are selected from an aromatic or heteroaromatic ring system which has 5 to 30 aromatic ring atoms and may be substituted in each case by one or more R¹ radicals, where R and R′ radicals may be bonded to one another and hence form a ring. More preferably, R and R′, if R′ is not H or D, are the same or different at each instance and are an aromatic or heteroaromatic ring system which has 5 to 18 aromatic ring atoms and may be substituted by one or more R¹ radicals.

In one embodiment of the invention, R and R′, if R′ is not H or D, are the same or different at each instance and are an aromatic or heteroaromatic ring system which has 5 to 18 aromatic ring atoms and has at least one R¹ radical other than H and D in ortho position to the bond to the nitrogen atom.

Suitable aromatic or heteroaromatic ring systems in the arylamino compound, or suitable R and R′ groups in formula (1) when R′ is not H or D, are the same or different at each instance and are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, phenanthrene or triphenylene, each of which may be substituted by one or more R¹ radicals. Also suitable are arylamino compounds in which R and R′ together with the nitrogen atom to which they are bonded form a carbazole, indenocarbazole or indolocarbazole, each of which may be substituted by one or more R¹ radicals.

Examples of suitable aromatic or heteroaromatic ring systems in the arylamino compound, or of suitable R and R′ groups when R′ is not H or D, are the groups of the following formulae R-1 to R-75:

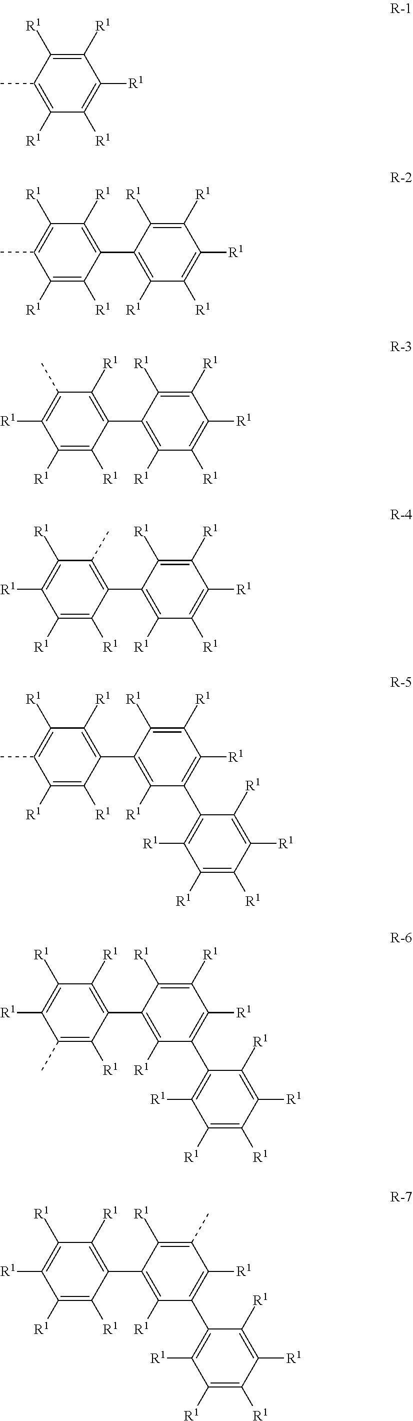

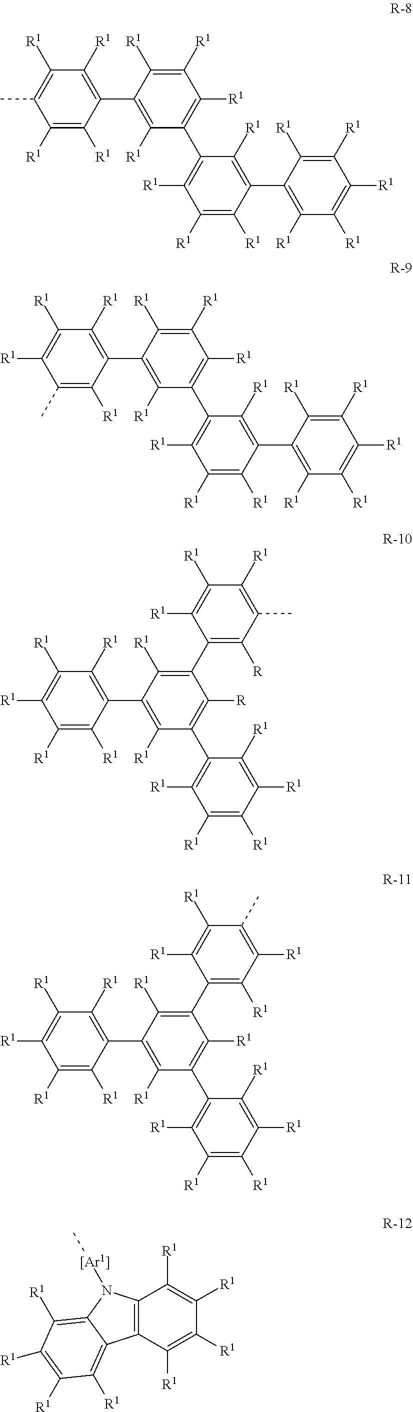

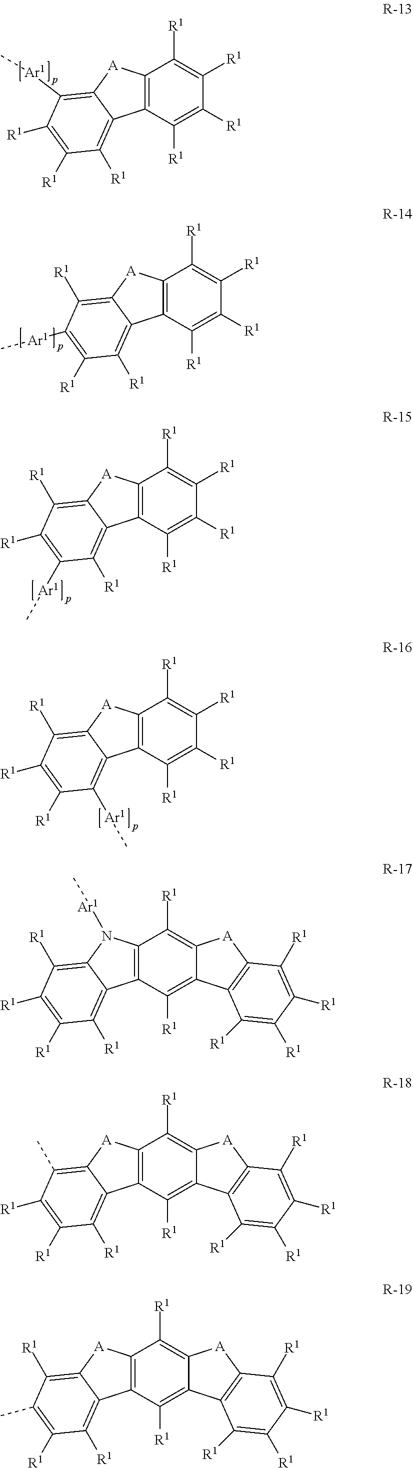

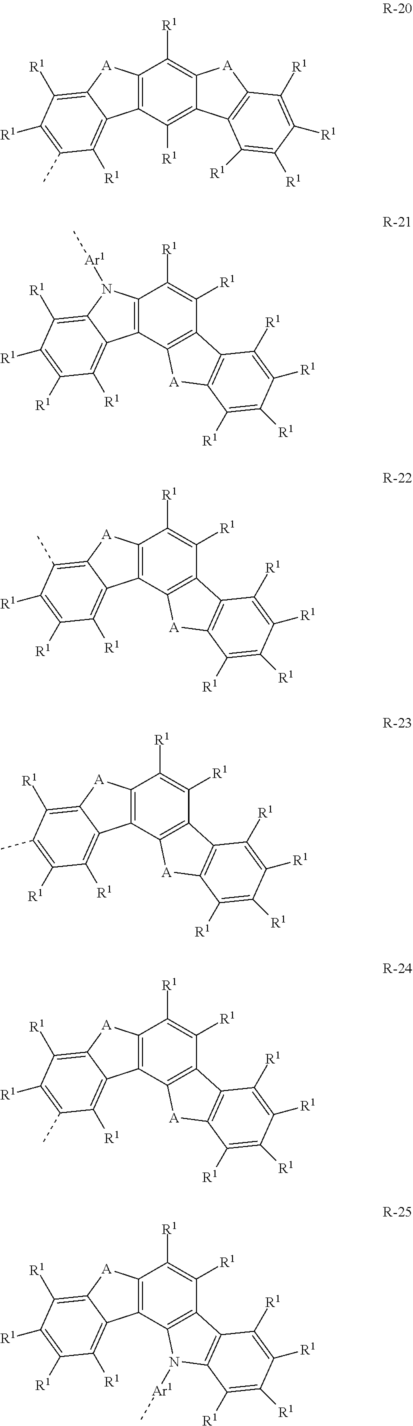

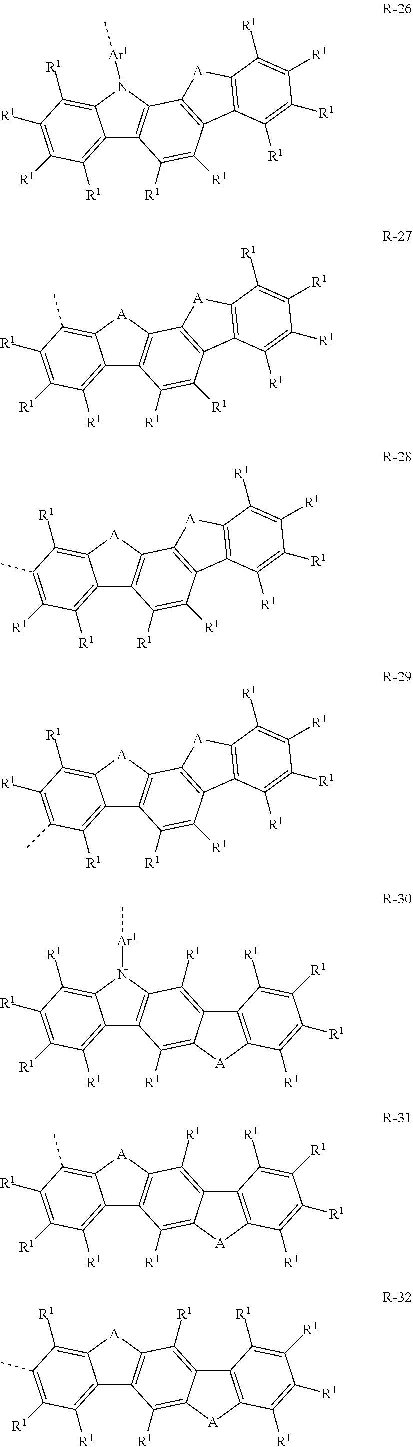

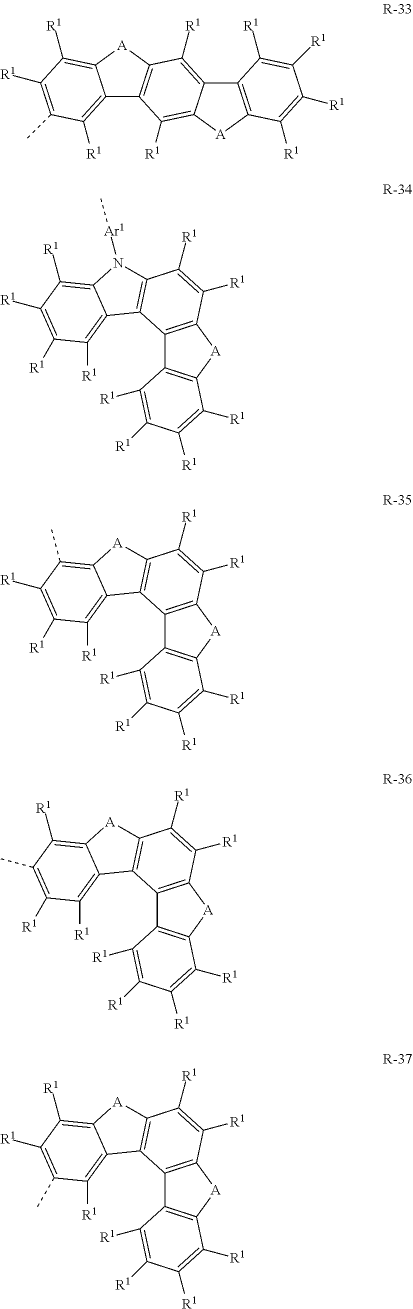

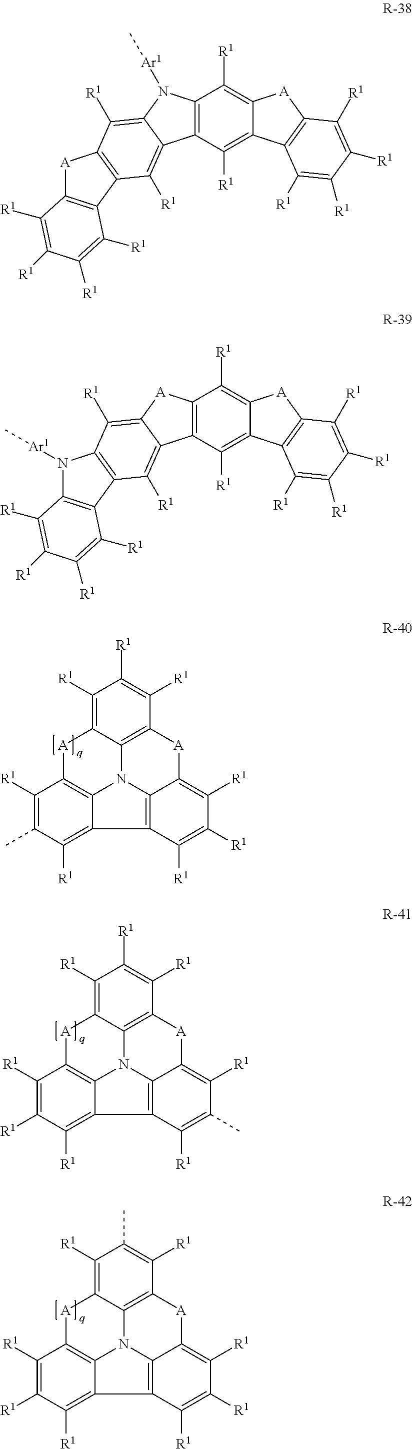

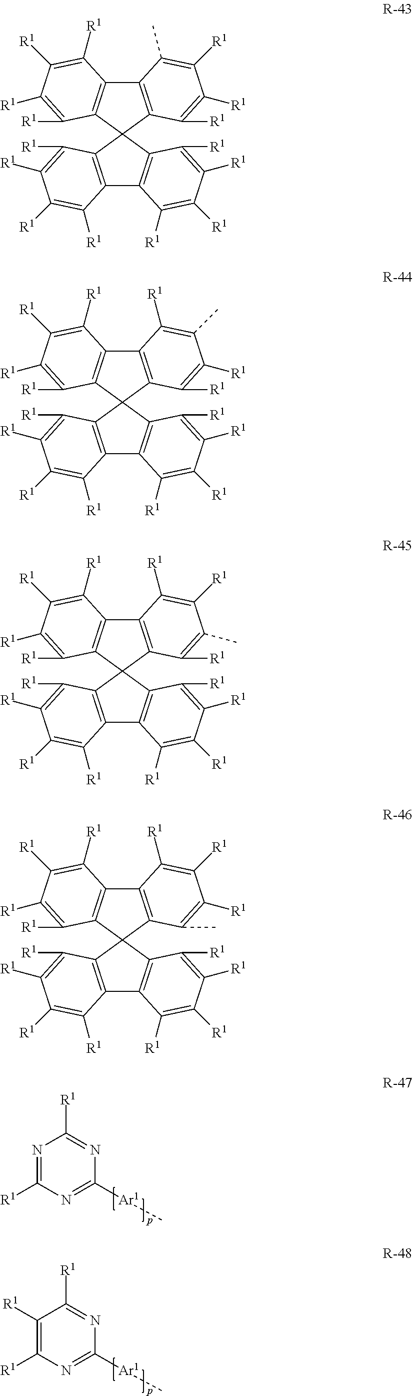

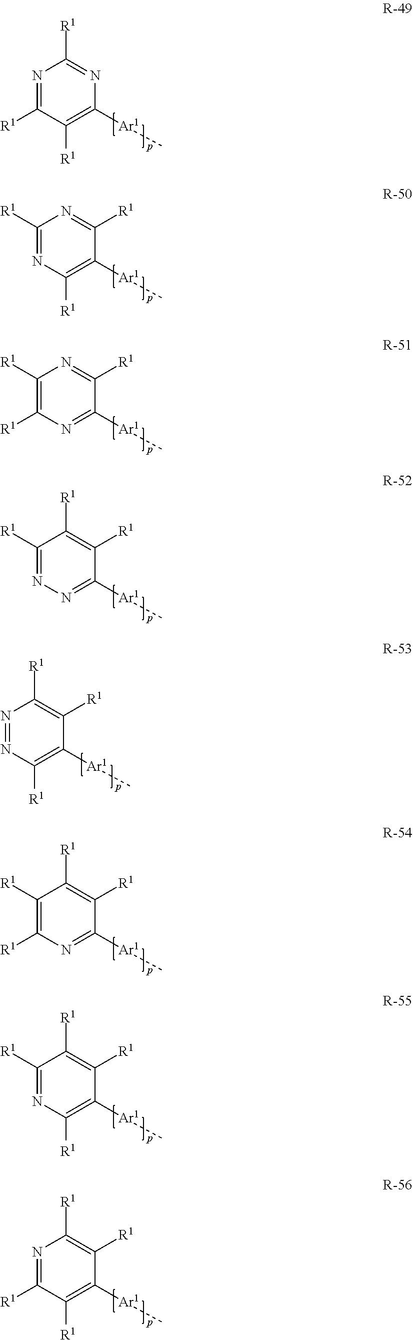

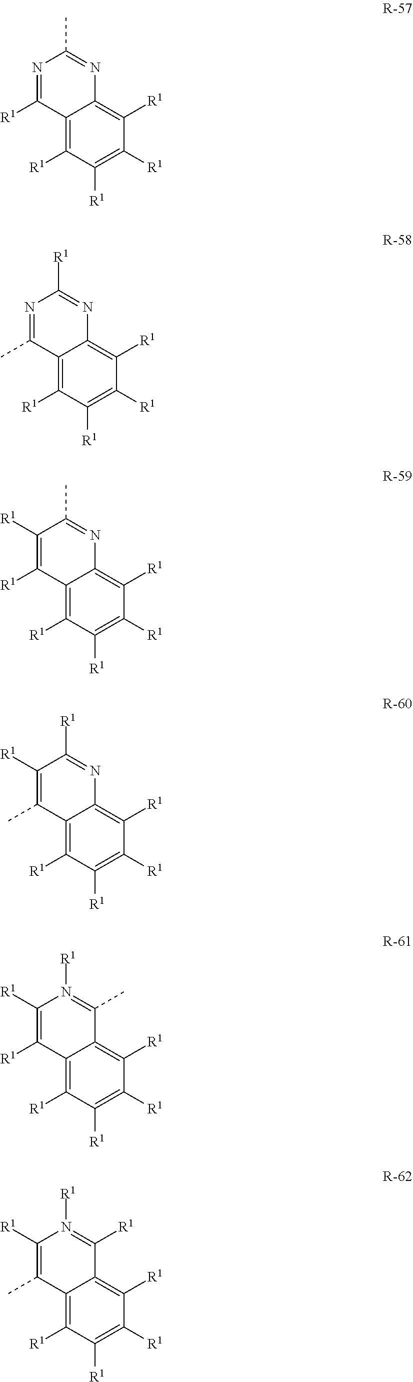

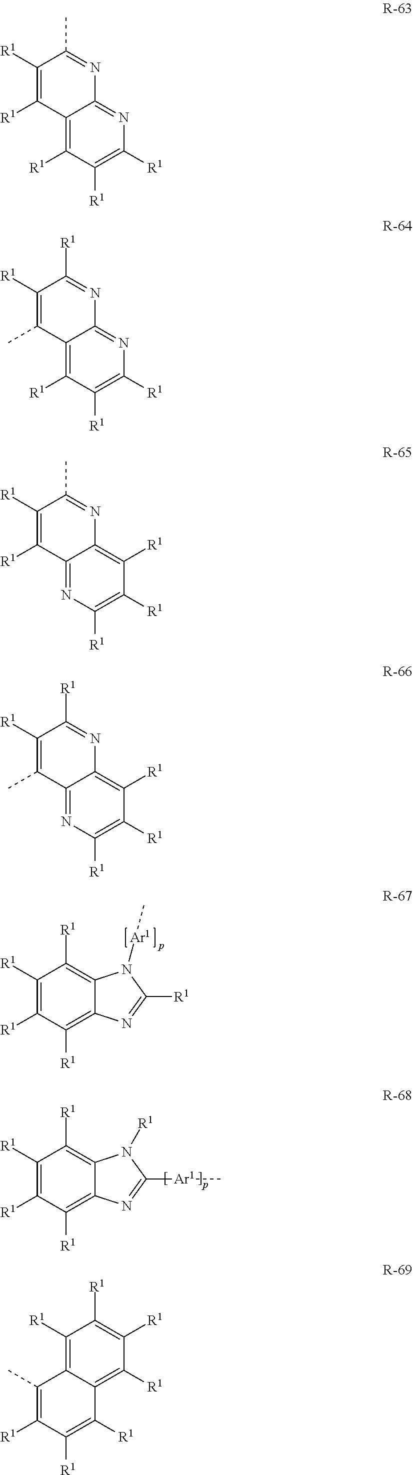

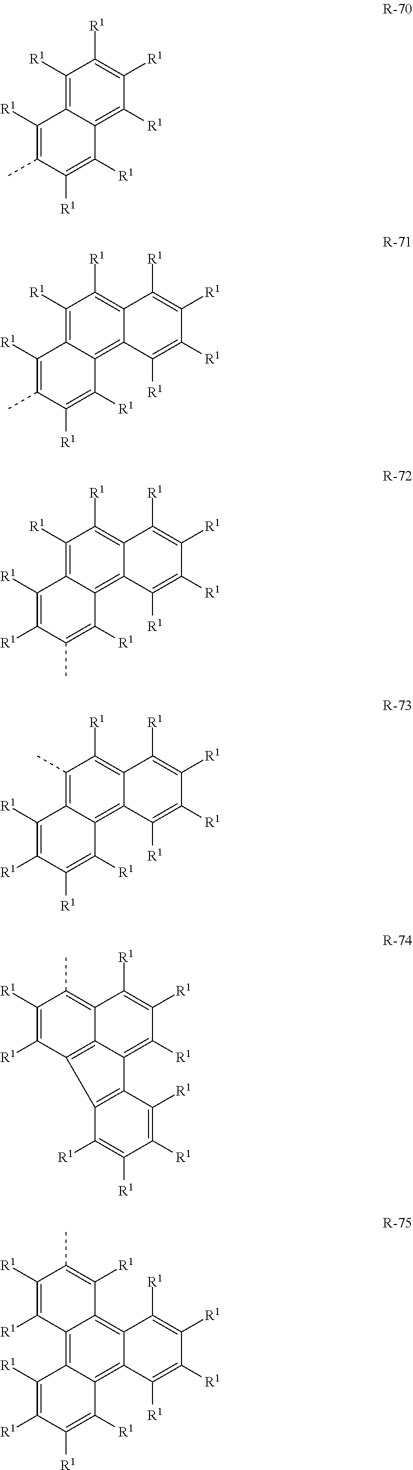

where R¹ has the definitions given above, the dotted bond represents the bond to the nitrogen atom, and in addition:

Ar¹ is the same or different at each instance and is a bivalent aromatic or heteroaromatic ring system which has 6 to 18 aromatic ring atoms and may be substituted in each case by one or more R¹ radicals;

A is the same or different at each instance and is C(R¹)₂, NR¹, O or S;

p is 0 or 1, where p=0 means that the Ar¹ group is absent and that the corresponding aromatic or heteroaromatic group is bonded directly to the nitrogen atom;

q is 0 or 1, where q=0 means that no A group is bonded at this position and R¹ radicals are bonded to the corresponding carbon atoms instead.

Preference is given here to the structures R-1 to R-46 and R-69 to R-75.

Preferred amines are the compounds listed in the following table:

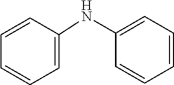 [SMILES: c1ccc(Nc2ccccc2)cc1]

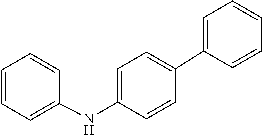 [SMILES: c1ccc(Nc2ccc(-c3ccccc3)cc2)cc1]

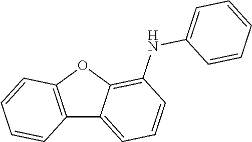 [SMILES: c1ccc(Nc2cccc3c2oc2ccccc23)cc1]

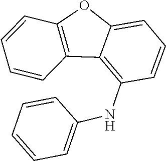 [SMILES: c1ccc(Nc2cccc3oc4ccccc4c23)cc1]

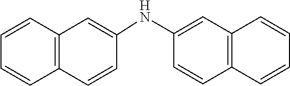 [SMILES: c1ccc2cc(Nc3ccc4ccccc4c3)ccc2c1]

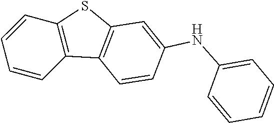 [SMILES: c1ccc(Nc2ccc3c(c2)sc2ccccc23)cc1]

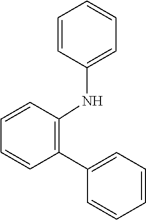 [SMILES: c1ccc(Nc2ccccc2-c2ccccc2)cc1]

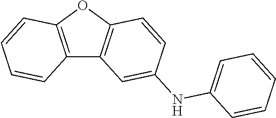 [SMILES: c1ccc(Nc2ccc3oc4ccccc4c3c2)cc1]

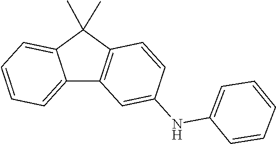 [SMILES: CC1(C)c2ccccc2-c2cc(Nc3ccccc3)ccc21]

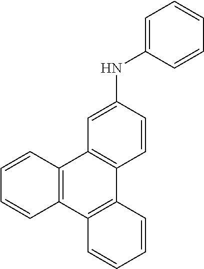 [SMILES: c1ccc(Nc2ccc3c4ccccc4c4ccccc4c3c2)cc1]

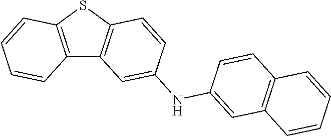 [SMILES: c1ccc2cc(Nc3ccc4sc5ccccc5c4c3)ccc2c1]

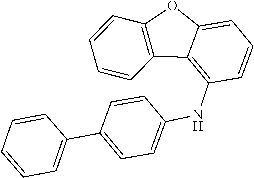 [SMILES: c1ccc(-c2ccc(Nc3cccc4oc5ccccc5c34)cc2)cc1]

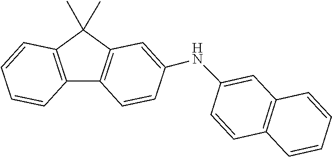 [SMILES: CC1(C)c2ccccc2-c2ccc(Nc3ccc4ccccc4c3)cc21]

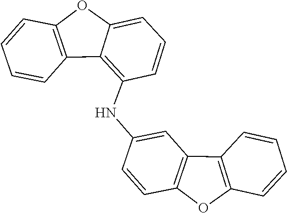 [SMILES: c1ccc2c(c1)oc1ccc(Nc3cccc4oc5ccccc5c34)cc12]

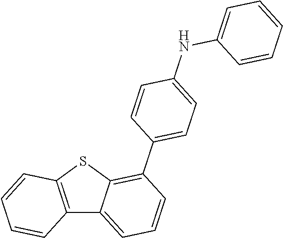 [SMILES: c1ccc(Nc2ccc(-c3cccc4c3sc3ccccc34)cc2)cc1]

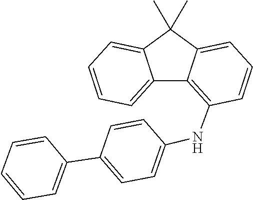 [SMILES: CC1(C)c2ccccc2-c2c(Nc3ccc(-c4ccccc4)cc3)cccc21]

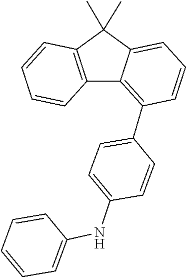 [SMILES: CC1(C)c2ccccc2-c2c(-c3ccc(Nc4ccccc4)cc3)cccc21]

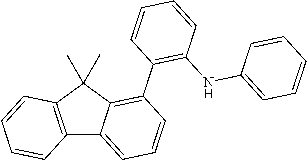 [SMILES: CC1(C)c2ccccc2-c2cccc(-c3ccccc3Nc3ccccc3)c21]

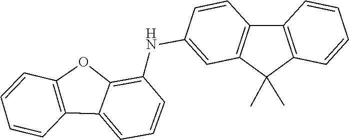 [SMILES: CC1(C)c2ccccc2-c2ccc(Nc3cccc4c3oc3ccccc34)cc21]

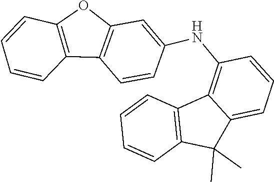 [SMILES: CC1(C)c2ccccc2-c2c(Nc3ccc4c(c3)oc3ccccc34)cccc21]

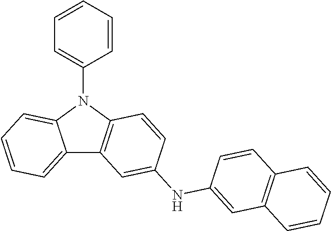 [SMILES: c1ccc(-n2c3ccccc3c3cc(Nc4ccc5ccccc5c4)ccc32)cc1]

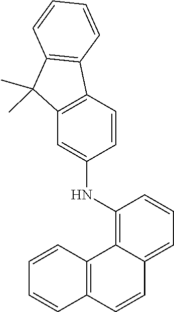 [SMILES: CC1(C)c2ccccc2-c2ccc(Nc3cccc4ccc5ccccc5c34)cc21]

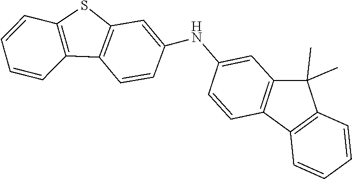 [SMILES: CC1(C)c2ccccc2-c2ccc(Nc3ccc4c(c3)sc3ccccc34)cc21]

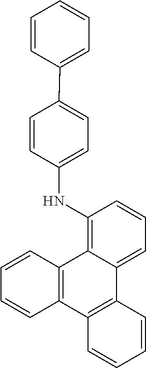 [SMILES: c1ccc(-c2ccc(Nc3cccc4c5ccccc5c5ccccc5c34)cc2)cc1]

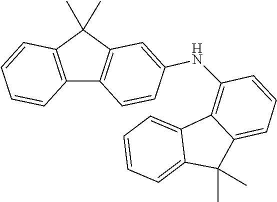 [SMILES: CC1(C)c2ccccc2-c2ccc(Nc3cccc4c3-c3ccccc3C4(C)C)cc21]

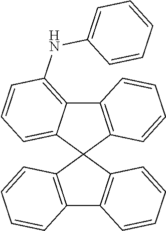 [SMILES: c1ccc(Nc2cccc3c2-c2ccccc2C32c3ccccc3-c3ccccc32)cc1]

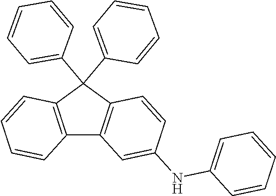 [SMILES: c1ccc(Nc2ccc3c(c2)-c2ccccc2C3(c2ccccc2)c2ccccc2)cc1]

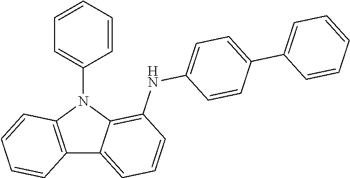 [SMILES: c1ccc(-c2ccc(Nc3cccc4c5ccccc5n(-c5ccccc5)c34)cc2)cc1]

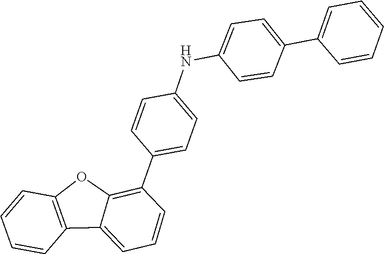 [SMILES: c1ccc(-c2ccc(Nc3ccc(-c4cccc5c4oc4ccccc45)cc3)cc2)cc1]

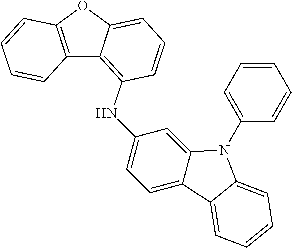 [SMILES: c1ccc(-n2c3ccccc3c3ccc(Nc4cccc5oc6ccccc6c45)cc32)cc1]

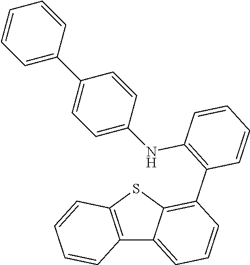 [SMILES: c1ccc(-c2ccc(Nc3ccccc3-c3cccc4c3sc3ccccc34)cc2)cc1]

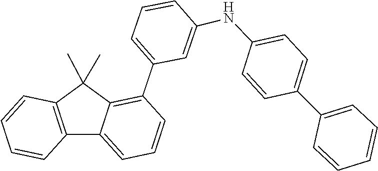 [SMILES: CC1(C)c2ccccc2-c2cccc(-c3cccc(Nc4ccc(-c5ccccc5)cc4)c3)c21]

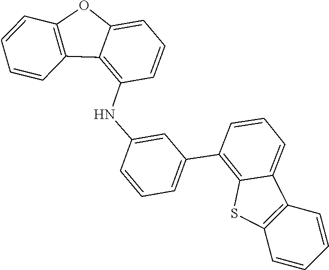 [SMILES: c1cc(Nc2cccc3oc4ccccc4c23)cc(-c2cccc3c2sc2ccccc23)c1]

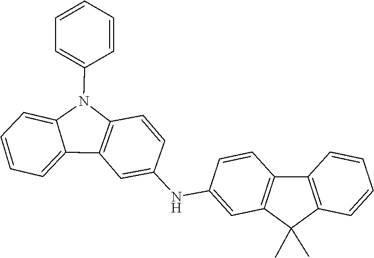 [SMILES: CC1(C)c2ccccc2-c2ccc(Nc3ccc4c(c3)c3ccccc3n4-c3ccccc3)cc21]

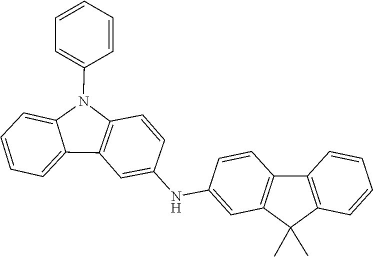 [SMILES: CC1(C)c2ccccc2-c2ccc(Nc3ccc4c(c3)c3ccccc3n4-c3ccccc3)cc21]

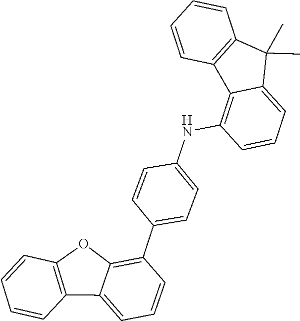 [SMILES: CC1(C)c2ccccc2-c2c(Nc3ccc(-c4cccc5c4oc4ccccc45)cc3)cccc21]

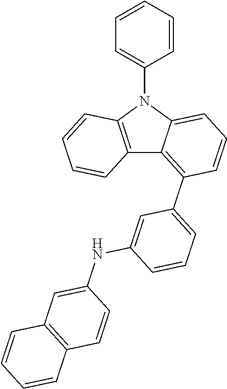 [SMILES: c1ccc(-n2c3ccccc3c3c(-c4cccc(Nc5ccc6ccccc6c5)c4)cccc32)cc1]

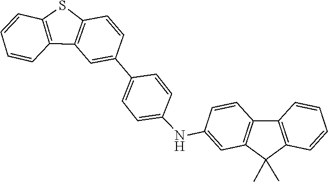 [SMILES: CC1(C)c2ccccc2-c2ccc(Nc3ccc(-c4ccc5sc6ccccc6c5c4)cc3)cc21]

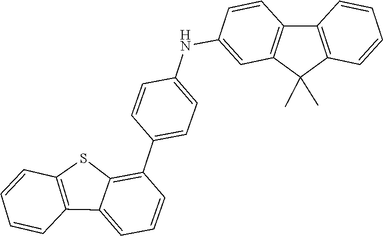 [SMILES: CC1(C)c2ccccc2-c2ccc(Nc3ccc(-c4cccc5c4sc4ccccc45)cc3)cc21]

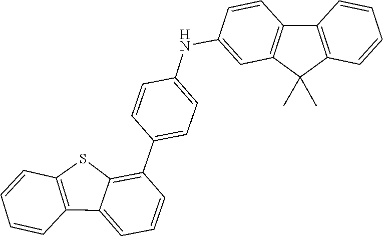 [SMILES: CC1(C)c2ccccc2-c2ccc(Nc3ccc(-c4cccc5c4sc4ccccc45)cc3)cc21]

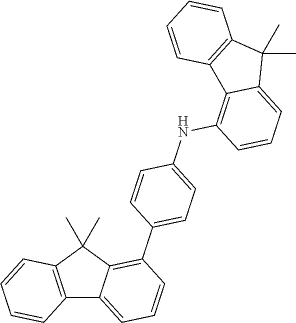 [SMILES: CC1(C)c2ccccc2-c2c(Nc3ccc(-c4cccc5c4C(C)(C)c4ccccc4-5)cc3)cccc21]

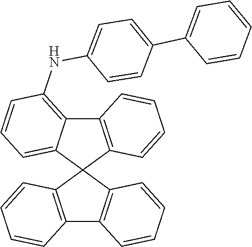 [SMILES: c1ccc(-c2ccc(Nc3cccc4c3-c3ccccc3C43c4ccccc4-c4ccccc43)cc2)cc1]

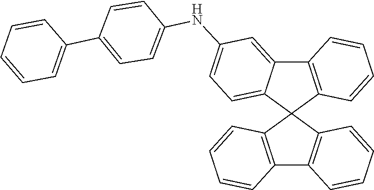 [SMILES: c1ccc(-c2ccc(Nc3ccc4c(c3)-c3ccccc3C43c4ccccc4-c4ccccc43)cc2)cc1]

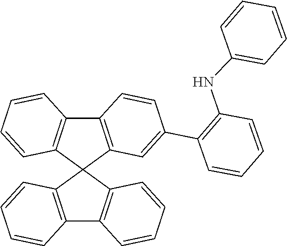 [SMILES: c1ccc(Nc2ccccc2-c2ccc3c(c2)C2(c4ccccc4-c4ccccc42)c2ccccc2-3)cc1]

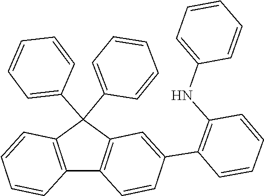 [SMILES: c1ccc(Nc2ccccc2-c2ccc3c(c2)C(c2ccccc2)(c2ccccc2)c2ccccc2-3)cc1]

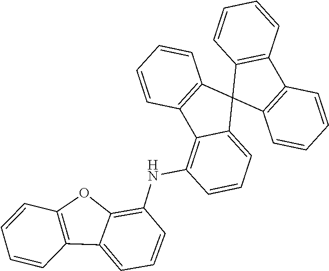 [SMILES: c1ccc2c(c1)-c1ccccc1C21c2ccccc2-c2c(Nc3cccc4c3oc3ccccc34)cccc21]

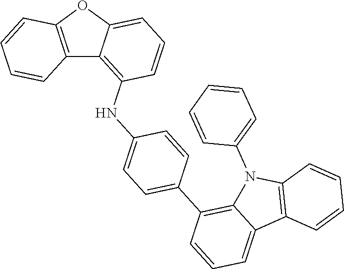 [SMILES: c1ccc(-n2c3ccccc3c3cccc(-c4ccc(Nc5cccc6oc7ccccc7c56)cc4)c32)cc1]

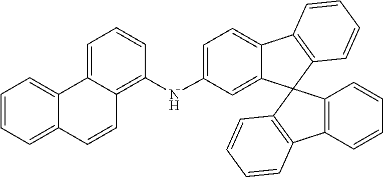 [SMILES: c1ccc2c(c1)-c1ccccc1C21c2ccccc2-c2ccc(Nc3cccc4c3ccc3ccccc34)cc21]

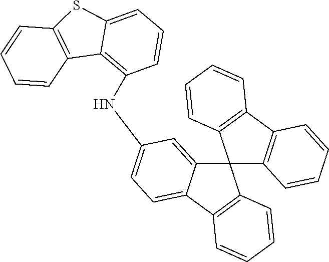 [SMILES: c1ccc2c(c1)-c1ccccc1C21c2ccccc2-c2ccc(Nc3cccc4sc5ccccc5c34)cc21]

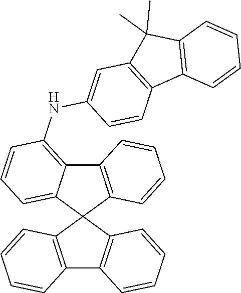 [SMILES: CC1(C)c2ccccc2-c2ccc(Nc3cccc4c3-c3ccccc3C43c4ccccc4-c4ccccc43)cc21]

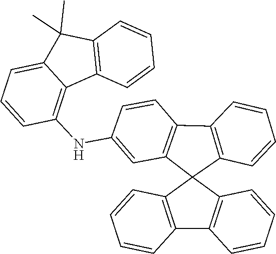 [SMILES: CC1(C)c2ccccc2-c2c(Nc3ccc4c(c3)C3(c5ccccc5-c5ccccc53)c3ccccc3-4)cccc21]

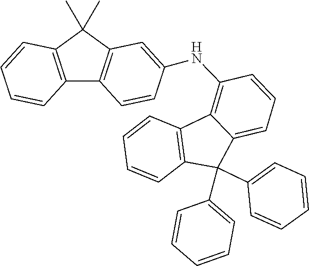 [SMILES: CC1(C)c2ccccc2-c2ccc(Nc3cccc4c3-c3ccccc3C4(c3ccccc3)c3ccccc3)cc21]

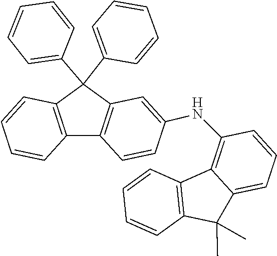 [SMILES: CC1(C)c2ccccc2-c2c(Nc3ccc4c(c3)C(c3ccccc3)(c3ccccc3)c3ccccc3-4)cccc21]

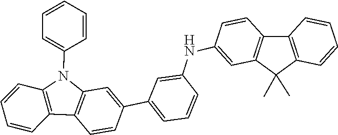 [SMILES: CC1(C)c2ccccc2-c2ccc(Nc3cccc(-c4ccc5c6ccccc6n(-c6ccccc6)c5c4)c3)cc21]

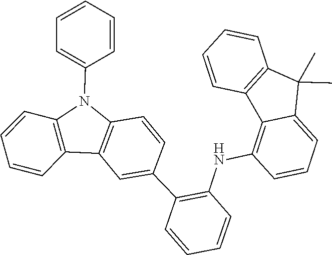 [SMILES: CC1(C)c2ccccc2-c2c(Nc3ccccc3-c3ccc4c(c3)c3ccccc3n4-c3ccccc3)cccc21]

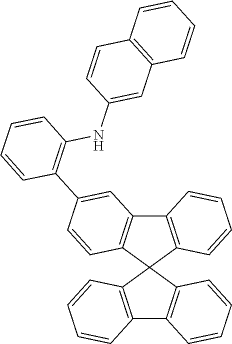 [SMILES: c1ccc(-c2ccc3c(c2)-c2ccccc2C32c3ccccc3-c3ccccc32)c(Nc2ccc3ccccc3c2)c1]

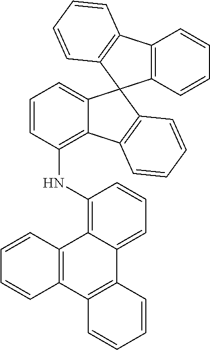 [SMILES: c1ccc2c(c1)-c1ccccc1C21c2ccccc2-c2c(Nc3cccc4c5ccccc5c5ccccc5c34)cccc21]

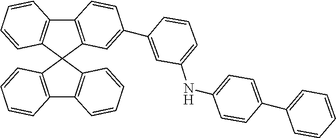 [SMILES: c1ccc(-c2ccc(Nc3cccc(-c4ccc5c(c4)C4(c6ccccc6-c6ccccc64)c4ccccc4-5)c3)cc2)cc1]

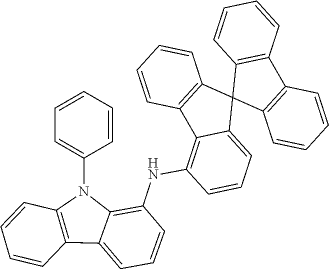 [SMILES: c1ccc(-n2c3ccccc3c3cccc(Nc4cccc5c4-c4ccccc4C54c5ccccc5-c5ccccc54)c32)cc1]

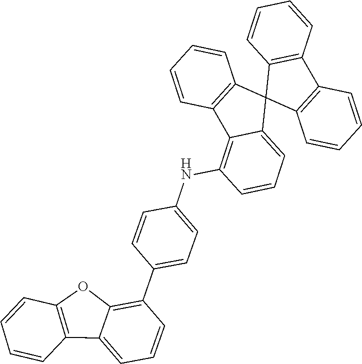 [SMILES: c1ccc2c(c1)-c1ccccc1C21c2ccccc2-c2c(Nc3ccc(-c4cccc5c4oc4ccccc45)cc3)cccc21]

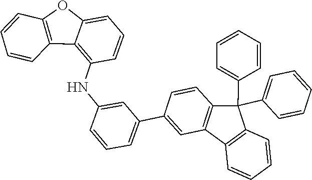 [SMILES: c1ccc(C2(c3ccccc3)c3ccccc3-c3cc(-c4cccc(Nc5cccc6oc7ccccc7c56)c4)ccc32)cc1]

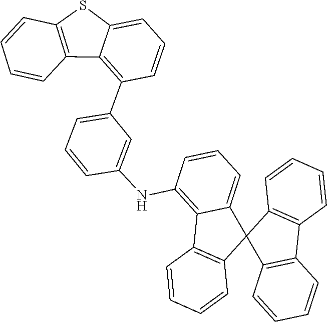 [SMILES: c1cc(Nc2cccc3c2-c2ccccc2C32c3ccccc3-c3ccccc32)cc(-c2cccc3sc4ccccc4c23)c1]

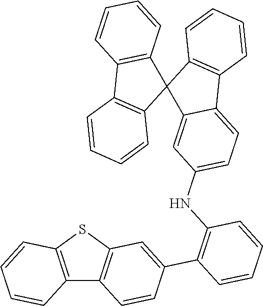 [SMILES: c1ccc(-c2ccc3c(c2)sc2ccccc23)c(Nc2ccc3c(c2)C2(c4ccccc4-c4ccccc42)c2ccccc2-3)c1]

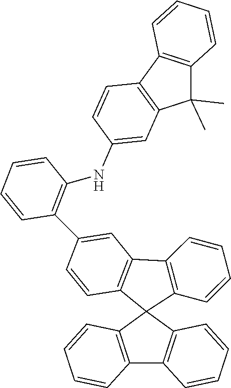 [SMILES: CC1(C)c2ccccc2-c2ccc(Nc3ccccc3-c3ccc4c(c3)-c3ccccc3C43c4ccccc4-c4ccccc43)cc21]

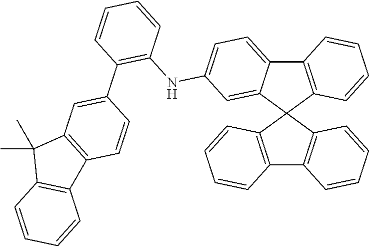 [SMILES: CC1(C)c2ccccc2-c2ccc(-c3ccccc3Nc3ccc4c(c3)C3(c5ccccc5-c5ccccc53)c3ccccc3-4)cc21]

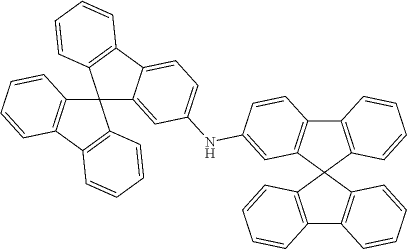 [SMILES: c1ccc2c(c1)-c1ccccc1C21c2ccccc2-c2ccc(Nc3ccc4c(c3)C3(c5ccccc5-c5ccccc53)c3ccccc3-4)cc21]

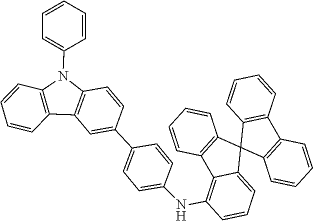 [SMILES: c1ccc(-n2c3ccccc3c3cc(-c4ccc(Nc5cccc6c5-c5ccccc5C65c6ccccc6-c6ccccc65)cc4)ccc32)cc1]

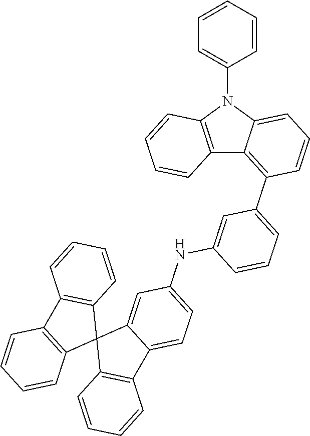 [SMILES: c1ccc(-n2c3ccccc3c3c(-c4cccc(Nc5ccc6c(c5)C5(c7ccccc7-c7ccccc75)c5ccccc5-6)c4)cccc32)cc1]

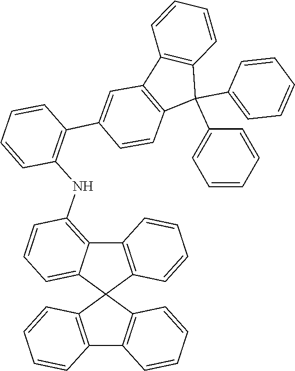 [SMILES: c1ccc(C2(c3ccccc3)c3ccccc3-c3cc(-c4ccccc4Nc4cccc5c4-c4ccccc4C54c5ccccc5-c5ccccc54)ccc32)cc1]

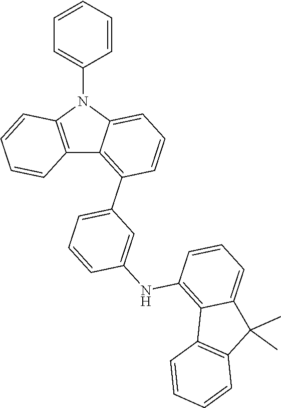 [SMILES: CC1(C)c2ccccc2-c2c(Nc3cccc(-c4cccc5c4c4ccccc4n5-c4ccccc4)c3)cccc21]

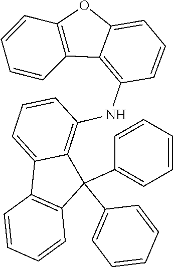 [SMILES: c1ccc(C2(c3ccccc3)c3ccccc3-c3cccc(Nc4cccc5oc6ccccc6c45)c32)cc1]

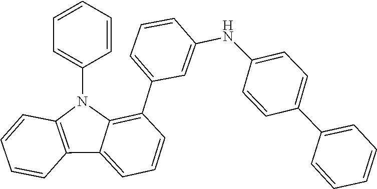 [SMILES: c1ccc(-c2ccc(Nc3cccc(-c4cccc5c6ccccc6n(-c6ccccc6)c45)c3)cc2)cc1]

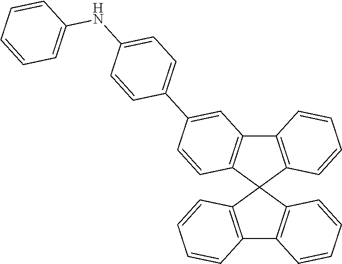 [SMILES: c1ccc(Nc2ccc(-c3ccc4c(c3)-c3ccccc3C43c4ccccc4-c4ccccc43)cc2)cc1]

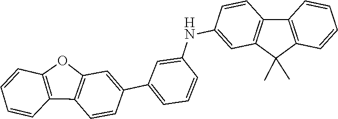 [SMILES: CC1(C)c2ccccc2-c2ccc(Nc3cccc(-c4ccc5c(c4)oc4ccccc45)c3)cc21]

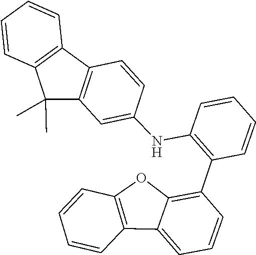 [SMILES: CC1(C)c2ccccc2-c2ccc(Nc3ccccc3-c3cccc4c3oc3ccccc34)cc21]

The leaving group in the aryl compound, or the X group in the compound of the formula (2), i.e. Ar—(X)ₙ, is preferably selected from the group consisting of optionally substituted alkylsulfonate, optionally substituted arylsulfonate, halide and diazonium. Examples of optionally substituted alkylsulfonates are trifluoromethylsulfonate (triflate, CF₃SO₃—) and methylsulfonate (mesylate, CH₃SO₃—). Examples of optionally substituted arylsulfonates are phenylsulfonate (C₆H₅—SO₃—) and tolylsulfonate (tosylate, CH₃—C₆H₄—SO₃—). Examples of halides are Cl, Br and I. The leaving group, or the X group, is preferably the same or different at each instance and is selected from the group consisting of triflate, phenylsulfonate and tosylate, more preferably triflate.

The index n in the compound of the formula (2) is preferably 1, 2, 3, 4 or 5, more preferably 1 or 2 and most preferably 1.

The aromatic or heteroaromatic ring system in the aryl compound, or the Ar group in formula (2), is preferably an aromatic or heteroaromatic ring system which has 5 to 30 aromatic ring atoms and may be substituted by one or more R¹ radicals. The aryl or heteroaryl group to which the leaving group X is bonded is preferably not an electron-deficient group. The Ar group is therefore preferably a purely aromatic ring system or an electron-rich heteroaromatic ring system. An electron-deficient heteroaryl group in the context of the present invention is a six-membered heteroaryl group containing at least one nitrogen atom, or a five-membered heteroaryl group containing at least two heteroatoms selected from N, O and S, where at least one heteroatom is N. It is possible for further aryl or heteroaryl groups to be fused onto these heteroaryl groups in each case. By contrast, an electron-rich heteroaryl group in the context of the present invention is a five-membered heteroaryl group containing exactly one heteroatom selected from O, S and N, where the nitrogen is substituted and where further aryl groups may be fused onto the five-membered ring. Examples of electron-rich heteroaryl groups in the context of the present invention are dibenzofuran, dibenzothiophene, carbazole, indenocarbazole and indolocarbazole.

Suitable aromatic or heteroaromatic ring systems in the aryl compound, or suitable Ar groups in formula (2), are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, phenanthrene or triphenylene, each of which may be substituted by one or more R¹ radicals.

Further preferably, the aromatic or heteroaromatic ring system in the aryl compound, or Ar in formula (2), is an aromatic or heteroaromatic ring system which has 10 to 30 aromatic ring atoms and has at least two aromatic or heteroaromatic rings fused to one another, where one aromatic or heteroaromatic ring is fused onto the other aromatic ring in an ortho position to the bond to the leaving group or to the bond to X. This also applies, for example, to dibenzofuran or dibenzothiophene each bearing a leaving group in the 1 position, or to carbazole bearing a leaving group in the 4 position.

Examples of suitable aromatic or heteroaromatic ring systems in the aryl compound or of suitable Ar groups are the groups of the following formulae

Ar-1 to Ar-53:

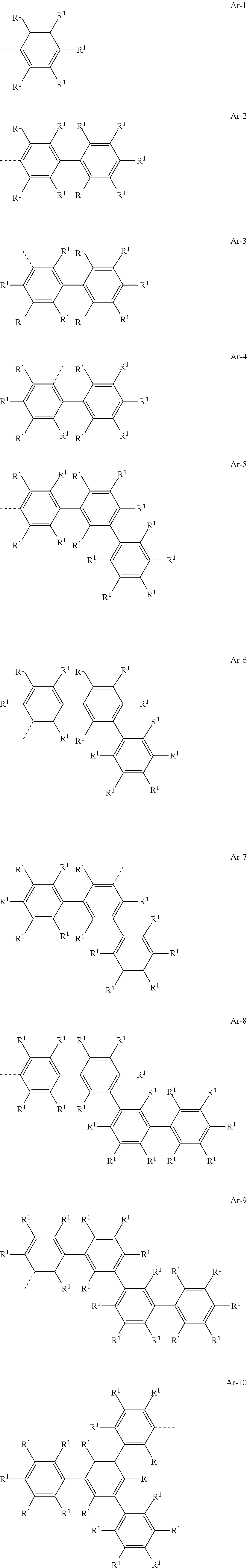

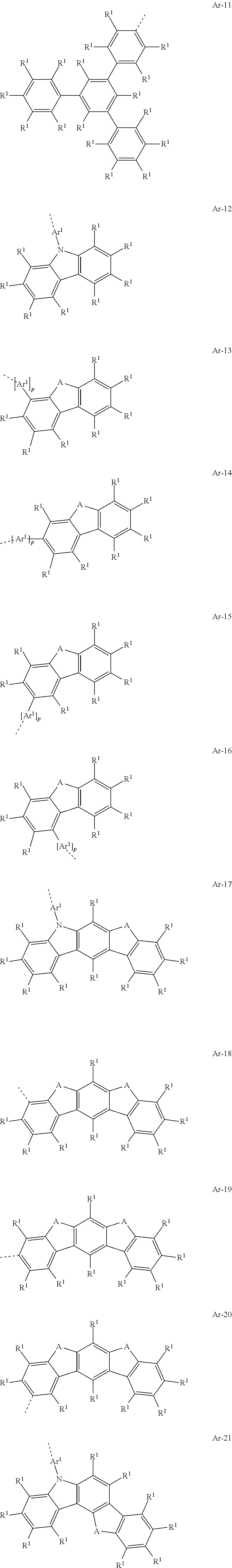

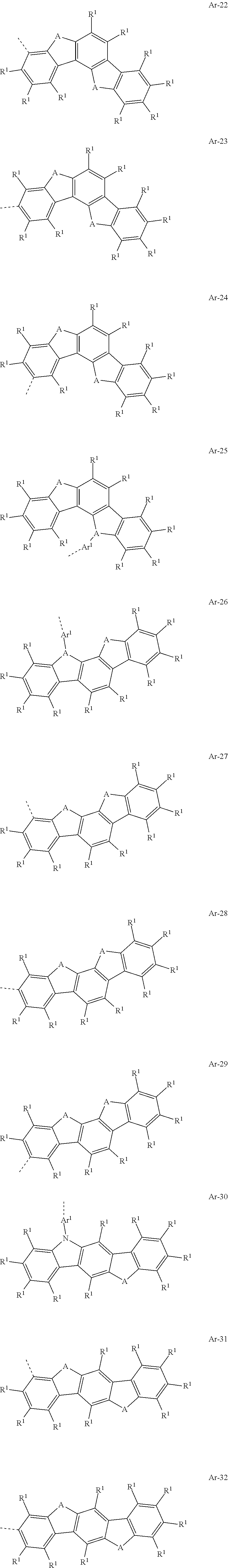

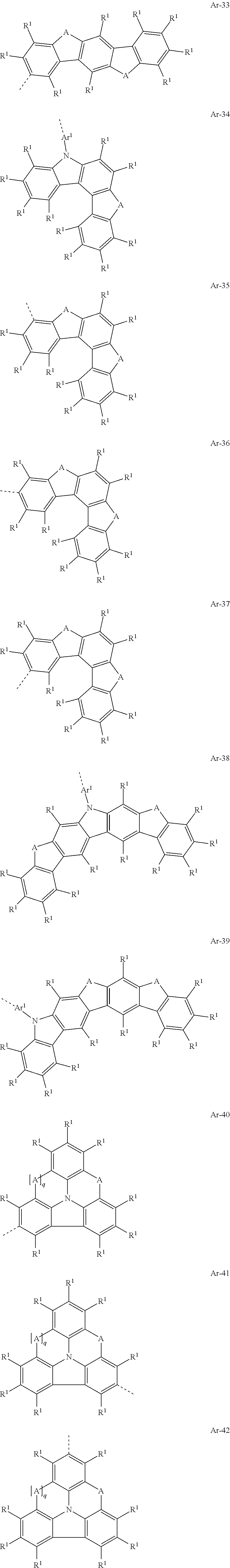

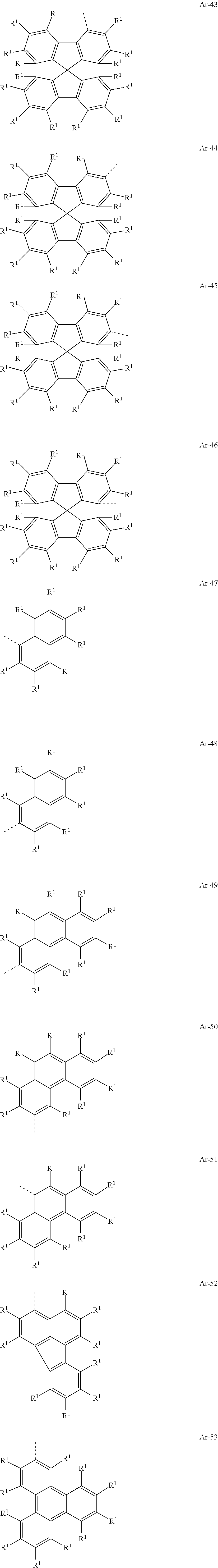

where R¹ has the definitions given above, the dotted bond represents the bond to the leaving group, i.e. X in the compound Ar—(X)ₙ, or the bond to the nitrogen atom in the reaction product, and in addition,

Ar¹ is the same or different at each instance and is a bivalent aromatic or heteroaromatic ring system which has 6 to 18 aromatic ring atoms and may be substituted in each case by one or more R¹ radicals;

A is the same or different at each instance and is C(R¹)₂, NR¹, O or S;

p is 0 or 1, where p=0 means that the Ar¹ group is absent and that the corresponding aromatic or heteroaromatic group is bonded directly to the leaving group or the X group;

q is 0 or 1, where q=0 means that no A group is bonded at this position and R¹ radicals are bonded to the corresponding carbon atoms instead.

When the abovementioned groups for R, R′ or Ar have two or more A groups, possible options for these include all combinations from the definition of A. Preferred embodiments in that case are those in which one A group is NR¹ and the other A group is C(R¹)₂ or in which both A groups are NR¹ or in which both A groups are O.

When A is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R² radicals. Preference is given to phenyl, biphenyl, terphenyl and quaterphenyl having bonding patterns as listed above for Ar-1 to Ar-11, where these structures, rather than by R¹, may be substituted by one or more R² radicals, but are preferably unsubstituted.

When A is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

The aryl compounds Ar—(X)ₙ here may also contain sterically demanding Ar groups, and likewise Ar groups substituted by sterically demanding R¹ groups, for example in the ortho position to the bond to the leaving group X, since the process of the invention also leads to good yields with sterically demanding aryl compounds Ar—(X)ₙ.

Preferably, R¹ is the same or different at each instance and is selected from H, D, F, CN, Si(R²)₃, N(R²)₂ or a straight-chain alkyl or alkoxy group having 1 to 10 carbon atoms or a branched or cyclic alkyl or alkoxy group having 3 to 10 carbon atoms, where the abovementioned groups may each be substituted by one or more R² radicals and where one or more CH₂ groups in the abovementioned groups may be replaced by —R²C═CR²— or —O—, or an aromatic or heteroaromatic ring system which has 5 to 24 aromatic ring atoms and may be substituted in each case by one or more R² radicals; at the same time, it is possible for two or more R¹ radicals to be joined to one another and hence form a ring. More preferably, R¹ is the same or different at each instance and is selected from H, D, F, CN, N(R²)₂ or a straight-chain alkyl group having 1 to 5 carbon atoms or a branched or cyclic alkyl group having 3 to 6 carbon atoms, where the abovementioned groups may each be substituted by one or more R² radicals, or an aromatic or heteroaromatic ring system which has 6 to 14 aromatic ring atoms and may be substituted in each case by one or more R² radicals; at the same time, it is possible for two or more R¹ radicals to be joined to one another and hence form a ring.

Preferably, R² is the same or different at each instance and is selected from H, D, F, CN, Si(R³)₃, N(R³)₂ or a straight-chain alkyl or alkoxy group having 1 to 10 carbon atoms or a branched or cyclic alkyl or alkoxy group having 3 to 10 carbon atoms, where the abovementioned groups may each be substituted by one or more R³ radicals and where one or more CH₂ groups in the abovementioned groups may be replaced by —R³C═CR³— or —O—, or an aromatic or heteroaromatic ring system which has 5 to 24 aromatic ring atoms and may be substituted in each case by one or more R³ radicals; at the same time, it is possible for two or more R² radicals to be joined to one another and hence form a ring. More preferably, R² is the same or different at each instance and is selected from H, D, F, CN, N(R³)₂ or a straight-chain alkyl group having 1 to 5 carbon atoms or a branched or cyclic alkyl group having 3 to 6 carbon atoms, where the abovementioned groups may each be substituted by one or more R³ radicals, or an aromatic or heteroaromatic ring system which has 6 to 14 aromatic ring atoms and may be substituted in each case by one or more R³ radicals; at the same time, it is possible for two or more R² radicals to be joined to one another and hence form a ring.

R³ is preferably the same or different at each instance and is H, D, F, an alkyl group having 1 to 5 carbon atoms or an aromatic ring system having 6 to 12 aromatic ring atoms.

The compounds obtained as reaction products are characterized in that, in the aryl compound, an Ar—N bond replaces the bond to the leaving group or the bond to X, where N denotes the nitrogen atom of the arylamino compound. Preferred process products of the process of the invention are thus combinations of the aryl compound and arylamino compound that are specified as preferred above.

According to the invention, the reaction is conducted in the presence of lithium tert-butoxide (LiOtBu) as base. The base is preferably added to the mixture at the start of the reaction.

The base is preferably used in an amount of 0.5 to 10 equivalents, based on the molar amount of aryl compound used. Particular preference is given to using 0.6 to 6 equivalents, very particular preference to using 1 to 4 equivalents, and greatest preference to using 2 to 3 equivalents, of base. If the aryl compound contains more than one leaving group, the base is used in accordance with the molar amount of leaving groups in the aryl compound used.

In the process of the invention, a palladium compound is used as catalyst. This can be added either at the start of the reaction or at a later juncture. It is preferable when the catalyst is added only after addition of the base to the mixture has concluded. The catalyst may alternatively already be present in the mixture when the base is added. The term “catalyst” in the context of the present application shall be understood to mean either a species that is actually catalytically active or a catalyst precursor that forms the catalytically active species in the reaction mixture. Preferred catalysts in the context of the present application are homogeneous catalysts, i.e. catalysts dissolved in the reaction medium. Catalysts used may generally be those compounds as used in the prior art for palladium-catalyzed C—N coupling reactions. These are known in principle to the person skilled in the art of organic synthesis.

The catalyst is preferably used in an amount of 0.001 to 10.0 mol %, more preferably 0.01 to 5.0 mol %, even more preferably 0.1 to 3.0 mol %, especially 0.5 to 2.5 mol %, based on the aryl compound. If the aryl compound contains more than one leaving group, the catalyst is added in accordance with the molar amount of leaving groups in the aryl compound used.

The catalyst comprises palladium and one or more ligands. The catalyst may be added in the form of a single compound comprising both the palladium and one or more ligands. Alternatively, the catalyst may be formed in situ in the reaction mixture from separately added palladium compound and ligand.

Suitable palladium compounds that may be used as independent catalysts without additional ligands are selected from Pd(PPh₃)₄, Pd-iPr-cinnamyl-Cl CX31 (CAS No. 884879-23-6), Pd-SiPR-cinnamyl-Cl CX32 (CAS No. 884879-24-7), Pd-PEPPSI-iPR (CAS No. 905459-27-0) and Pd-PEPPSI-iPent (CAS No. 1158652-41-5).

Suitable palladium compounds as catalyst constituents that are used with additional ligands are selected from PdCl₂ (CAS No. 7647-10-1), Pd(OAc)₂ (CAS No. 3375-31-3), (CH₃CN)2PdCl2 (CAS No. 14592-56-4), bis(dibenzylideneacetone)palladium (Pd(dba)2) (CAS No. 32005-36-0) and tris(dibenzylideneacetone)dipalladium (Pd₂(dba)₃) (CAS No. 51364-51-3). Preference is given to Pd(OAc)₂.

The ligands are preferably selected from monodentate and oligodentate ligands, especially monodentate ligands. Preferred ligands are selected from phosphines, phosphites, amines, aminophosphines and N-heterocyclic carbenes. Suitable phosphines and amines for use as ligands are disclosed in WO 2011/008725, WO 2006/074315 and U.S. Pat. No. 6,307,087. Suitable N-heterocyclic carbenes for use as ligands are disclosed in CA 2556850. Preference is given to monodentate and bidentate phosphines, especially monodentate phosphines.

Suitable phosphines as ligands are selected from dicyclohexylphosphino-2′,6′-dimethoxybiphenyl (SPhos, CAS No. 657408-07-6), dicyclohexylphosphino-2′,6′-diisopropoxybiphenyl (RuPhos, CAS No. 787618-22-8), di-tert-butyl(2′,4′,6′-triisopropyl-3,6-dimethoxybiphenyl-2-yl)phosphine (tBuBrettPhos, CAS No. 1160861-53-9), dicyclohexyl(2′,4′,6′-triisopropyl-3,6-dimethoxybiphenyl-2-yl)phosphine (BrettPhos, CAS No. 1070663-78-3), trimethylphosphine (CAS No. 594-09-2), triethylphosphine (CAS No. 554-70-1), tripropylphosphine (CAS No. 2234-97-1), triisopropylphosphine (CAS No. 6476-36-4), tributylphosphine (CAS No. 998-40-3), tri-tert-butylphosphine (CAS No. 13716-12-6), triphenylphosphine (CAS No. 603-35-0), di-tert-butylchlorophosphine (CAS No. 13716-10-4), trimethyl phosphite (CAS No. 121-45-9), triethyl phosphite (CAS No. 122-52-1), tripropyl phosphite (CAS No. 923-99-9), triisopropyl phosphite (CAS No. 116-17-6), tributyl phosphite (CAS No. 102-85-2), tricyclohexyl phosphite (CAS No. 15205-58-0), tri(o-tolyl)phosphine (CAS No. 6163-58-2), triisopropylphosphine (CAS No. 6476-36-4), tricyclohexylphosphine (CAS No. 2622-14-2), 2,2′-bis(diphenylphosphino)-1,1′-binaphthyl (BINAP, CAS No. 98327-87-8), 1,2-bis(dimethylphosphino)ethane (CAS No. 23936-60-9), 1,2-bis(diethylphosphino)ethane (CAS No. 6411-21-8), 1,2-bis(dipropylphosphino)ethane (CAS No. 86071-87-6), 1,2-bis(diisopropylphosphino)ethane (CAS No. 87532-69-2), 1,2-bis(dibutylphosphino)ethane (CAS No. 4141-59-7), 1,2-bis(dicyclohexylphosphino)ethane (CAS No. 23743-26-2), 1,3-bis(dicyclohexylphosphino)propane (CAS No. 103099-52-1), 1,3-bis(diisopropylphosphino)propane (CAS No. 91159-11-4), 1,4-bis(diisopropylphosphino)butane (CAS No. 80499-19-0), 2,4-bis(dicyclohexylphosphino)pentane (CAS No. 96377-46-7) and 1,1′-bis(diphenylphosphino)ferrocene (dppf, CAS No. 12150-46-8). Further suitable ligands are Cy-JohnPhos (CAS No. 247940-06-3), CataCxium Pcy (CAS No. 672937-60-9), Aphos (CAS No. 932710-63-9), XantPhos (CAS No. 161265-03-8) and XPhos (CAS No. 564483-18-7). Preferred ligands are SPhos, XPhos and CPhos, more preferably SPhos.

The palladium compound and the phosphine ligand, in the case of monophosphines, are preferably used in a Pd:phosphine ratio in the range from 1:1 to 1:4, more preferably in the range from 1:1.5 to 1:2.5 and most preferably in the range from 1:1.8 to 1:2.2. In the case of biphosphines, the palladium compound and the phosphine ligand are preferably used in a Pd:phosphine ratio in the range from 1:0.5 to 1:2, more preferably in the range from 1:0.75 to 1:1.5, most preferably in the range from 1:0.9 to 1:1.1.

The process of the invention is preferably performed in the liquid phase in solution or suspension. According to the leaving group, the lithium salt, for example lithium triflate, precipitates out of the reaction mixture in the course of the reaction. It is possible here to use any aprotic organic solvent. Suitable solvents are those known to the person skilled in the art in the field of organic synthesis that are suitable for use in palladium-catalyzed C—N coupling reactions. What is meant more particularly by suitability for use in the reactions of the invention is that these are inert with respect to the reaction conditions. Particularly preferred solvents are selected from benzene, toluene, 1,2-xylene, 1,3-xylene, 1,4-xylene, mesitylene, tetrahydrofuran (THF), 1,4-dioxane, dimethoxyethane (dme) and bis(2-methoxyethyl) ether (diglyme). Preference is given to using anhydrous solvents. Very particular preference is given to toluene, 1,2-xylene, 1,3-xylene, 1,4-xylene and mesitylene, especially toluene.

The reaction can be performed under standard pressure or under elevated pressure.

In a preferred embodiment, the reaction is performed under inert gas atmosphere, for example under nitrogen or argon atmosphere. Particularly good results are achieved when very good inert conditions are consistently maintained, as exist, for example, in a glovebox.

The reaction time is typically between a few minutes and a few days, preferably between a few minutes and 100 h, more preferably between 15 minutes and 80 h and most preferably between 2 h and 30 h. The times stated are based on the overall duration of the reaction.

The reaction temperature is preferably between 0 and 300° C., more preferably between 20 and 200° C., most preferably between 60 and 150° C. The reaction temperatures stated are based on the main phase of the reaction, during which all components are present in the mixture and the C—N bond formation takes place. In a further preferred embodiment, the reaction is performed under reflux, such that the reaction temperature is determined by the solvent used. Therefore, a preferred reaction temperature is 100° C. when toluene is used as solvent, 144° C. in the case of 1,2-xylene, 139° C. in the case of 1,3-xylene, 138° C. in the case of 1,4-xylene, and 165° C. in the case of mesitylene.

Typically, the base is included in the initial charge together with the reactants, i.e. the arylamino compound and the aryl compound, in a solvent and inertized. In parallel to that, the catalyst solution is prepared, preferably using the same solvent which is also used in the reaction solution. The catalyst solution is then added to the reaction mixture, and the mixture is reacted at elevated temperature. Alternatively, the catalyst or the palladium compound and the ligand may also be added in solid form.

The general reaction regime and workup and the equipment and reaction vessels used are otherwise unspecific. The person skilled in the art, with regard to the working examples and drawing on his common art knowledge, will be able to select suitable embodiments. Especially suitable methods are those as known to the person skilled in the art in general for Hartwig-Buchwald couplings.

When the products obtained in the process of the invention are secondary arylamines, these are preferably used as intermediate for preparation of tertiary arylamines. When the products obtained in the process of the invention are tertiary arylamines, these are preferably used as functional materials in electronic devices. These electronic devices are selected, for example, from the group consisting of organic electroluminescent devices (OLEDs), organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs) and organic laser diodes (O-lasers). The process products obtained are preferably used as hole transport materials and/or as fluorescent emitter materials and/or as electron blocker materials and/or as exciton blocker materials and/or as matrix materials in electronic devices, especially in organic electroluminescent devices. However, other uses are also possible, depending on the skeleton of the products and the substituents present in addition to the amino group.

The present invention further provides for the use of LiOtBu as base in a palladium-catalyzed coupling reaction between an arylamino compound and an aryl compound. The arylamino compound and the aryl compound are defined as specified above.

The process of the invention, combined with a preceding Suzuki coupling, enables the selective functionalization of aromatic or heteroaromatic compounds. For example, it is possible, in an aryl compound substituted by two leaving groups, of which one leaving group is a halide and the other is a sulfonate, first to selectively react the halide with a boronic acid derivative in a Suzuki coupling and then, in a further step, the sulfonate in a C—N coupling reaction according to the present invention. Alternatively, it is possible first to selectively react the sulfonate with a boronic acid derivative in a Suzuki coupling and then, in a further step, to react the halide in a C—N coupling reaction according to the present invention. It is possible here to determine via the choice of catalyst system whether the sulfonate leaving group or the halide leaving group is selectively converted. For instance, a selective conversion of the halide leaving group, for example bromide, is obtained in the Suzuki coupling when tri-ortho-tolylphosphine is used as ligand, especially in a Pd:phosphine ratio in the order of magnitude of 1:4. In addition, a selective conversion of the sulfonate leaving group, for example triflate, is obtained in the Suzuki coupling when dppb (1,4-bis(diphenylphosphino)butane) is used as ligand, especially in a Pd:phosphine ratio in the order of magnitude of 1:4. Further suitable reaction conditions are conditions as typically used for Suzuki couplings, i.e., for example, the use of Pd2(dba)3 as palladium compound, Na₂CO₃ or K₂CO₃ as base and a mixture of toluene or another organic solvent and water as reaction medium.

The process of the invention has the following advantages over prior art processes:
1) The use of LiOtBu compared to other bases, especially compared to NaOtBu or KOtBu, leads to a distinct improvement in yield. This is particularly true when sulfonates, especially triflate, are used as leaving group. One feature of the process of the invention is that a high yield of product and low formation of by-products occur.
2) Even when sterically demanding arylamino compounds or sterically demanding aryl compounds are used as reactants, only a low level of by-product in the form of defunctionalization or coupling of the base used is observed in the process of the invention. This represents a distinct improvement over the use of NaOtBu or KOtBu as base.
3) The use of LiOtBu as base leads to a high purity of the compounds obtained. This is of major significance specifically for the use of the compounds as functional materials in electronic devices. The formation of by-products that have a detrimental effect on the performance data of the electronic device even in a small amount is suppressed here by the use of LiOtBu.
4) The process of the invention can also be performed on a scale as required for industrial use, i.e. on a scale up to several kilograms.
5) The process can be performed with a small amount of catalyst used and under mild reaction conditions.

The examples which follow serve for illustration and detailed description of the invention without any intention to restrict it thereby.

### EXAMPLES

The syntheses which follow, unless stated otherwise, are conducted under a protective gas atmosphere in dried solvents. The solvents and reagents can be purchased, for example, from Sigma-ALDRICH or ABCR. The respective figures in square brackets or the numbers quoted for individual compounds relate to the CAS numbers of the compounds known from the literature.

### Example 1

### General Procedure

The triflate, the amine and the base are weighed out under an argon atmosphere in a glovebox. After catalyst and ligand have been added, the solvent is added and the vessel closed. The reaction vessel is discharged from the glovebox and stirred in an oil bath at internal temperature 110° C. for 16 h. After cooling to room temperature, a sample is taken and analyzed by HPLC.

### Example 2

### Variation of the Base

In the reaction that follows, Pd-iPr-cinnamyl-CI CX31 (CAS No. 884879-23-6) is used as catalyst. The base used is LiOtBu according to the present invention, or NaOtBu or KOtBu as comparison according to the prior art. The results are collated in table 1. The reaction with LiOtBu as base leads to a distinctly better yield compared to NaOtBu or KOtBu, and a good yield is still obtained even when the amount of catalyst is reduced.

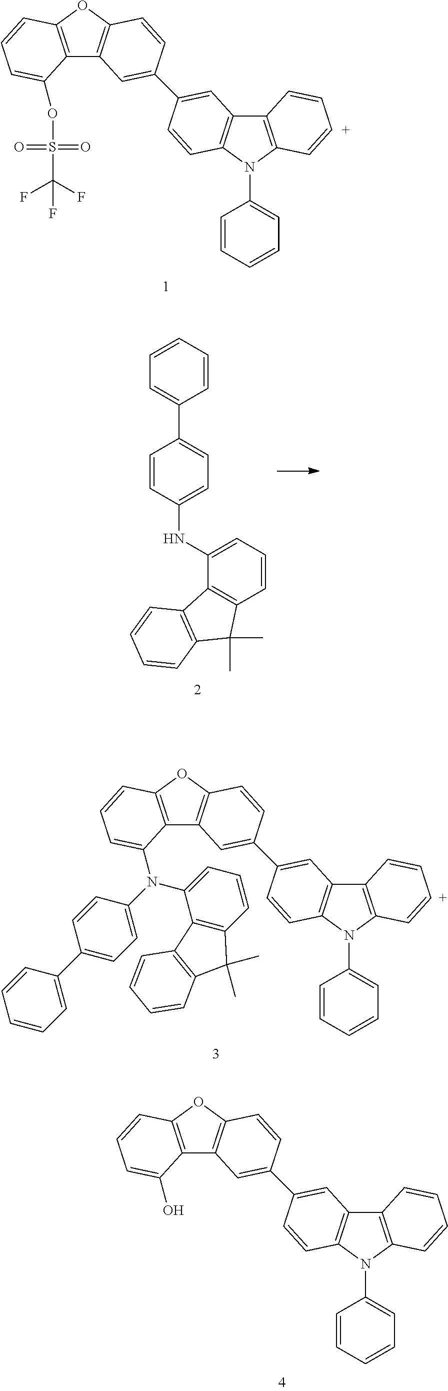 [SMILES: O=S(=O)(Oc1cccc2oc3ccc(-c4ccc5c(c4)c4c(n5-c5ccccc5)C=[C+]C=C4)cc3c12)C(F)(F)F ; CC1(C)c2ccccc2-c2c(Nc3ccc(-c4ccccc4)cc3)cccc21 ; CC1(C)c2ccccc2-c2c(N(c3ccc(-c4ccccc4)cc3)c3cccc4oc5ccc(-c6ccc7c(c6)c6c(n7-c7ccccc7)C=[C+]C=C6)cc5c34)cccc21 ; Oc1cccc2oc3ccc(-c4ccc5c(c4)c4ccccc4n5-c4ccccc4)cc3c12]

TABLE 1^{[a]}
Variation of the base with Pd-iPr-cinnamyl-CI CX31
as catalyst

| | [Pd] | Base | Y₁ | Y₂ | Y₃ | Y₄ |
|---|---|---|---|---|---|---|
| Ex. | [mol %] | (200 mol %) | [%] | [%] | [%] | [%] |
| 1 | 2 | NaOtBu | 0 | 12 | 58 | 25 |
| (comp.) | | | | | | |
| 2 | 2 | KOtBu | 0 | 28 | 1 | 52 |
| (comp.) | | | | | | |
| 3 | 2 | LiOtBu | 1 | 3 | 91 | 1 |
| 4 | 0.5 | LiOtBu | 11 | 17 | 40 | 30 |

^{[a]}Reaction conditions: 0.44 mmol 8-(9-pheny1-9H-carbazol-3-yl)dibenzofuran-1-yl trifluoromethanesulfonate
1, 0.48 mmol bipheny1-4-y1-(9,9-dimethy1-9H-fluoren-4-yl)amine
2, 8 ml toluene,
110° C., 16 h. Area percent determined by HPLC.

### Example 3

### Use of a Different Catalyst System

In this example, the reaction from example 2 is conducted with a different catalyst system, with variation of the amount of catalyst and base. The catalyst used for this purpose is a system composed of Pd(OAc)₂ and SPhos (table 2).

TABLE 2
Optimization of the experimental parameters for the
Pd(OAc)₂/SPhos/LiOtBu system.^{[a]}

| | Pd(OAc)₂ | SPhos | LiOtBu | Y₁ | Y₂ | Y₃ | Y₄ |
|---|---|---|---|---|---|---|---|
| Ex. | [mol %] | [mol %] | [mol %] | [%] | [%] | [%] | [%] |
| 1 | 1 | 2 | 220 | 2 | 6 | 82 | 8 |
| 2 | 1 | 2 | 150 | 8 | 3 | 75 | 8 |
| 3 | 1 | 2 | 110 | 25 | 3 | 57 | 12 |
| 4 | 0.5 | 1 | 220 | 2 | 8 | 74 | 14 |
| 5 | 1 | 1 | 220 | 2 | 6 | 83 | 5 |
| 6 | 1 | 1.5 | 220 | 1 | 8 | 82 | 6 |
| 7 | 2 | 4 | 200 | 0 | 3 | 94 | 0 |
| 8 | 2 | 4 | 250 | 0 | 3 | 95 | 0 |
| 9 | 2 | 4 | 300 | 0 | 3 | 95 | 0 |

^{[a]}Reaction conditions: 0.44 mmol 8-(9-pheny1-9H-carbazol-3-yl)dibenzofuran-1-y1 trifluoromethanesulfonate
1, 0.48 mmol bipheny1-4-y1-(9,9-dimethy1-9H-fluoren-4-yl)amine
2, 8 ml toluene,
110° C., 16 h. Area percent determined by HPLC.

### Example 4

### Use of Different Ligands and Different Substrates

In this example, the reaction is conducted with different ligands and different substrates, with retention of Pd(OAc)₂ as palladium source and LiOtBu as base (tables 3 and 4).

### a) Reaction of a Dibenzofuran Triflate With Bis(Para-Biphenyl)Amine

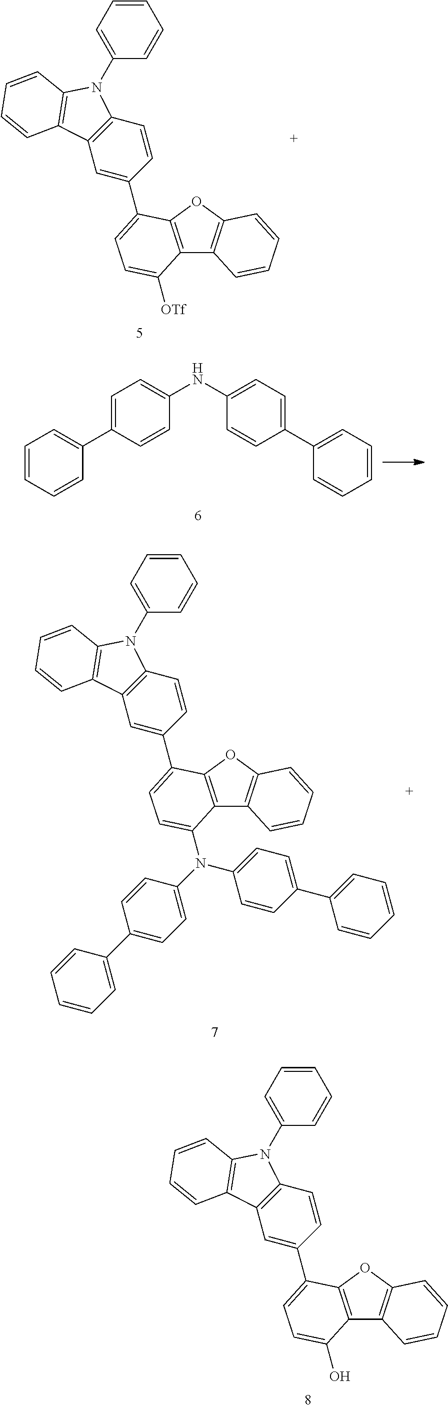 [SMILES: O=S(=O)(Oc1ccc(-c2ccc3c(c2)c2ccccc2n3-c2ccccc2)c2oc3ccccc3c12)C(F)(F)F ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc5c(c4)c4ccccc4n5-c4ccccc4)c4oc5ccccc5c34)cc2)cc1 ; Oc1ccc(-c2ccc3c(c2)c2ccccc2n3-c2ccccc2)c2oc3ccccc3c12]

TABLE 3^{[a]}
Variation of the ligand

| Ex. | Ligand | Y₅ [%] | Y₆ [%] | Y₇ [%] | Y₈ [%] |
|---|---|---|---|---|---|
| 1 | SPhos | 0.02 | 0.69 | 96.4 | — |
| | 4 mol % | | | | |
| 2 | XantPhos | 0.09 | 4.79 | 71.3 | — |
| | 2 mol % | | | | |
| 3 | CPhos | 0.54 | 1.09 | 95.8 | — |
| | 4 mol % | | | | |
| 4 | XPhos | 0.32 | 0.76 | 96.9 | — |
| | 4 mol % | | | | |

^{[a]}Reaction conditions: 0.60 mmol 4-(9-phenyl-9H-carbazol-3-yl)dibenzofuran-1-yl trifluoromethanesulfonate 5, 0.60 mmol bis(biphenyl-4-yl)amine 6, 250 mol % LiOtBu, 2 mol % Pd(OAc)₂, 6.0 ml toluene, 110°C., 16 h. Area percent determined by HPLC.

### b) Reaction of a Dibenzofuran Triflate With a Para-Biphenyl-4-Fluorenylamine

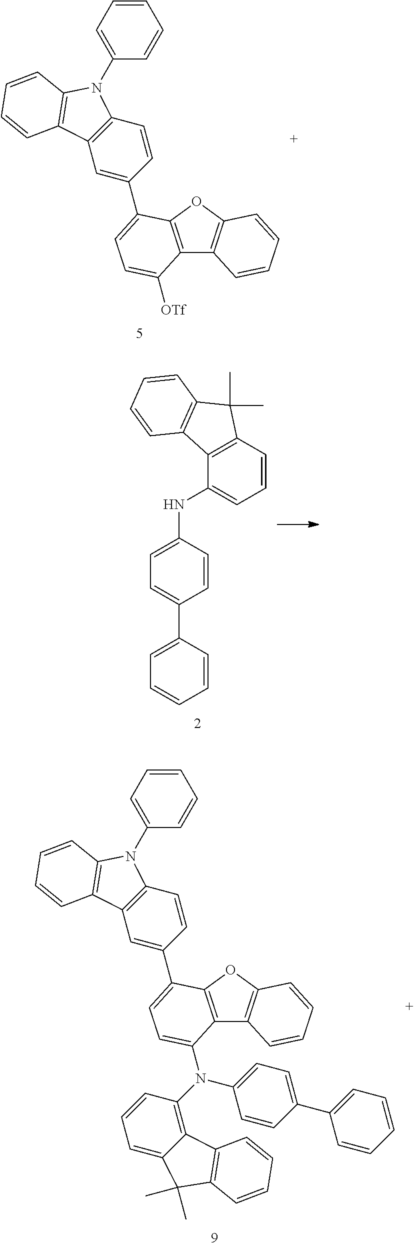 [SMILES: O=S(=O)(Oc1ccc(-c2ccc3c(c2)c2ccccc2n3-c2ccccc2)c2oc3ccccc3c12)C(F)(F)F ; CC1(C)c2ccccc2-c2c(Nc3ccc(-c4ccccc4)cc3)cccc21 ; CC1(C)c2ccccc2-c2c(N(c3ccc(-c4ccccc4)cc3)c3ccc(-c4ccc5c(c4)c4ccccc4n5-c4ccccc4)c4oc5ccccc5c34)cccc21]

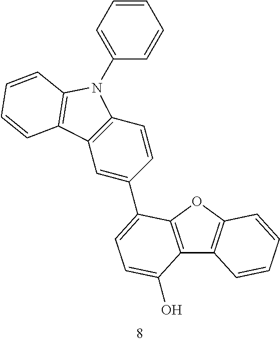 [SMILES: Oc1ccc(-c2ccc3c(c2)c2ccccc2n3-c2ccccc2)c2oc3ccccc3c12]

TABLE 4^{[a]}
Variation of the ligands

| Ex. | | Y₅ [%] | Y₂ [%] | Y₉ [%] | Y₈ [%] |
|---|---|---|---|---|---|
| 1 | SPhos | 1.37 | 4.71 | 84.0 | — |
| 2 | XPhos | 0.75 | 2.98 | 87.5 | — |

^{[a]}Reaction conditions: 0.35 mmol 4-(9-phenyl-9H-carbazol-3-yl)dibenzofuran-1-yl trifluoromethanesulfonate 5, 0.39 mmol biphenyl-4-yl-(9, 9-dimethyl-9H-fluoren-4-yl)amine 2, 220 mol % LiOtBu, 2 mol % Pd(OAc)₂, 4 mol % ligand, 6.0 ml toluene, 110° C., 16 h. Area percent determined by HPLC.

### Example 5

### Use of Different Substrates

In this example, the reaction conditions from table 3, entry 4, and table 4, entry 2, are applied to different substrates (table 5). For this purpose, in this example, phenyl triflate is reacted with ortho-biphenyl(phenyl)amine as a sterically demanding secondary amine. The reaction with LiOtBu as base leads to a significantly better yield here compared to NaOtBu.

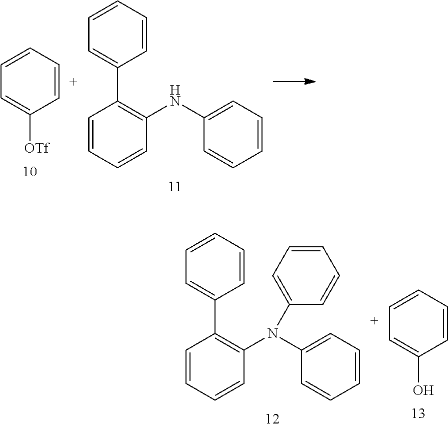

TABLE 5^{[a]}

| Ex. | Base | Y₁₀ [%] | Y₁₁ [%] | Y₁₂ [%] | Y₁₃ [%] |
|---|---|---|---|---|---|
| 1 | LiOtBu | 0 | 15.3 | 76.4 | — |
| 2 (comp.) | NaOtBu | 0 | 91.3 | 2.3 | — |

^{[a]}Reaction conditions: 0.86 mmol phenyl trifluoromethanesulfonate 10, 0.95 mmol biphenyl-2-yl(phenyl)amine 11, 220 mol % base, 2 mol % Pd(OAc)₂, 4 mol % XPhos, 5.7 ml toluene, 110° C., 16 h. Area percent determined by HPLC.

### Example 6

### Reaction of Phenyl Triflate With Carbazole

In this example, carbazole is used as a secondary amine, i.e. an amine in which the two aromatic groups are joined to one another (table 6). The reaction with LiOtBu as base leads to a distinctly better yield here compared to NaOtBu.

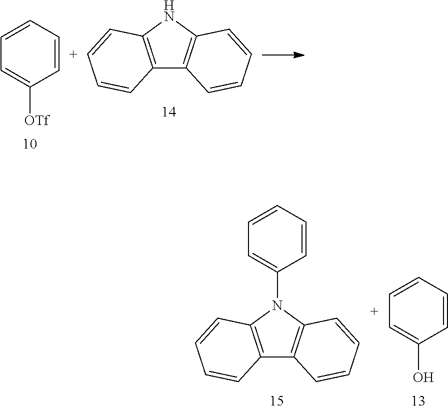

TABLE 6^{[a]}

| Ex. | Base | Y₁₀ [%] | Y₁₄ [%] | Y₁₅ [%] | Y₁₃ [%] |
|---|---|---|---|---|---|
| 1 | LiOtBu | 0 | — | 96.1 | — |
| 2 (comp.) | NaOtBu | 0 | — | 77.8 | — |

^{[a]}Reaction conditions: 0.86 mmol phenyl trifluoromethanesulfonate 10, 0.95 mmol carbazole 14, 220 mol % base, 2 mol % Pd(OAc)₂, 4 mol % XPhos, 5.7 ml toluene, 110° C., 16 h. Area percent determined by HPLC.

### Example 7

### Reaction of 1-Naphthyl Triflate With Secondary Amine

In this example, the aryl compound used is 1-naphthyl triflate, i.e. a compound having a fused aryl group (table 7). The reaction with LiOtBu as base leads to a significantly better yield here compared to NaOtBu.

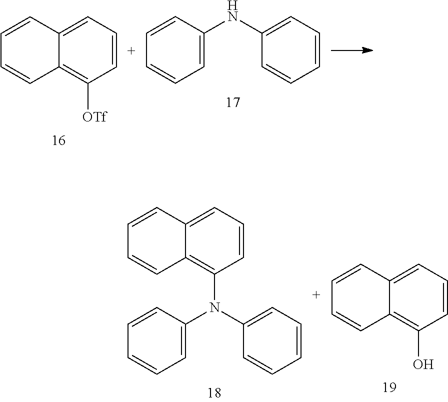

TABLE 7^{[a]}

| Ex. | Base | Y₁₆ [%] | Y₁₇ [%] | Y₁₈ [%] | Y₁₉ [%] |
|---|---|---|---|---|---|
| 1 | LiOtBu | 0.5 | 3.2 | 94.1 | 0.2 |
| 2 (comp.) | NaOtBu | 0 | 32.4 | 8.5 | 46.9 |

^{[a]}Reaction conditions: 0.70 mmol 1-naphthyl trifluoromethanesulfonate 16, 0.77 mmol diphenylamine 17, 220 mol % base, 2 mol % Pd(OAc)₂, 4 mol % XPhos, 5.7 ml toluene, 110° C., 16 h. Area percent determined by HPLC.

### Example 8

### Reaction of 1-Naphthyl Triflate With Sterically Demanding Secondary Amine

In this example, the aryl compound reacted is 1-naphthyl triflate, i.e. an aryl compound having a fused aryl group, with phenyl(ortho-biphenyl)amine as a sterically demanding secondary amine (table 8). The reaction with LiOtBu as base leads to a good yield here, whereas virtually no conversion to the tertiary amine at all is observed with NaOtBu as base.

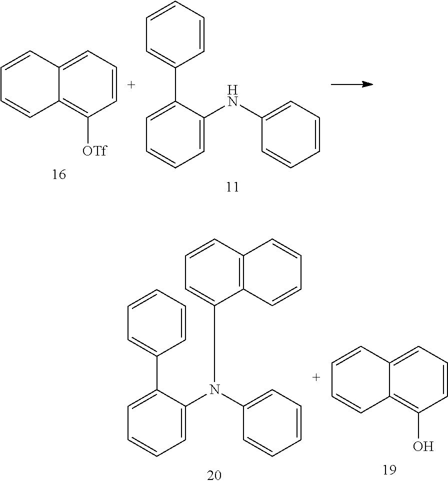

TABLE 8^{[a]}

| Ex. | Base | Y₁₆ [%] | Y₁₁ [%] | Y₂₀ [%] | Y₁₀ [%] |
|---|---|---|---|---|---|
| 1 | LiOtBu | 19.9 | 21.4 | 51.6 | 0 |
| 2 (comp.) | NaOtBu | 0.1 | 55.9 | 0.4 | 35.6 |

^{[a]}Reaction conditions: 0.70 mmol 1-naphthyl trifluoromethanesulfonate 16, 0.77 mmol biphenyl-2-yl(phenyl)amine 11, 220 mol % base, 2 mol % Pd(OAc)₂, 4 mol % XPhos, 5.7 ml toluene, 110° C., 16 h. Area percent determined by HPLC.

### Example 9

### Reaction of Sterically Demanding Triflate and Sterically Demanding Secondary Amine

In this example, ortho-biphenyl triflate as a sterically demanding aryl compound is reacted with phenyl(ortho-biphenyl)amine as a sterically demanding amine (table 9). The reaction with LiOtBu as base leads to a good yield here, whereas no conversion to the tertiary amine at all is observed with NaOtBu as base.

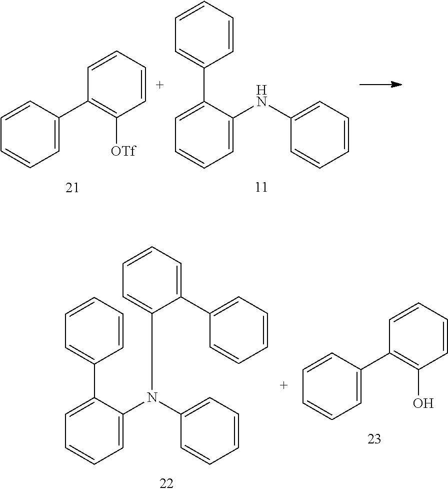

TABLE 9^{[a]}:

| Ex. | Base | Y₂₁ [%] | Y₁₁ [%] | Y₂₂ [%] | Y₂₃ [%] |
|---|---|---|---|---|---|
| 1 | LiOtBu | 3.3 | 29.9 | 21.2 | 2.5 |
| 2 (comp.) | NaOtBu | 0 | 63.6 | 0 | 33.7 |

^{[a]}Reaction conditions: 0.65 mmol biphenyl-2-yl trifluoromethanesulfonate 21, 0.72 mmol biphenyl-2-yl(phenyl)amine 11, 220 mol % base, 2 mol % Pd(OAc)₂, 4 mol % XPhos, 5.7 ml toluene, 110° C., 16 h. Area percent determined by HPLC.

### Example 10

### Reaction of Sterically Demanding Triflate With Secondary Amine

In this example, ortho-biphenyl triflate as a sterically demanding aryl compound is reacted with diphenylamine as secondary amine (table 10). The reaction with LiOtBu as base leads to a very good yield here, whereas no conversion to the tertiary amine at all is observed with NaOtBu as base.

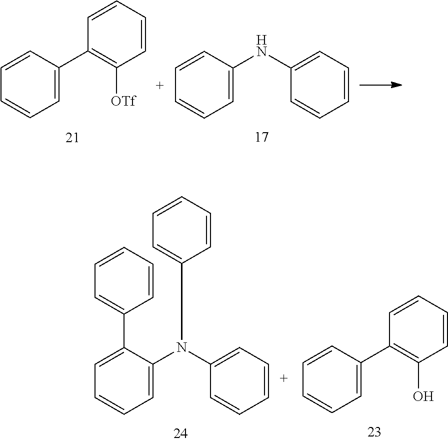

TABLE 10^{[a]}

| Ex. | Base | Y₂₁ [%] | Y₁₇ [%] | Y₂₄ [%] | Y₂₃ [%] |
|---|---|---|---|---|---|
| 1 | LiOtBu | 0 | 5.5 | 89.7 | 0.3 |
| 2 (comp.) | NaOtBu | 0 | 45.5 | 0 | 52.5 |

^{[a]}Reaction conditions: 0.65 mmol biphenyl-2-yl trifluoromethanesulfonate 21, 0.72 mmol diphenylamine 17, 220 mol % base, 2 mol % Pd(OAc)₂, 4 mol % XPhos, 5.7 ml toluene, 110° C., 16 h. Area percent determined by HPLC.

### Example 11

### Reaction of Sterically Demanding 9-Phenyl-9H-Carbazol-4-yl Trifluoromethanesulfonate With Carbazole

In this example, a carbazolyl derivative substituted by triflate as leaving group in the 4 position, as sterically demanding aryl compound, is reacted with carbazole as secondary amine (table 11). The reaction with LiOtBu as base leads to a good yield here, whereas no conversion to the C—N-coupled product at all is observed with NaOtBu as base.

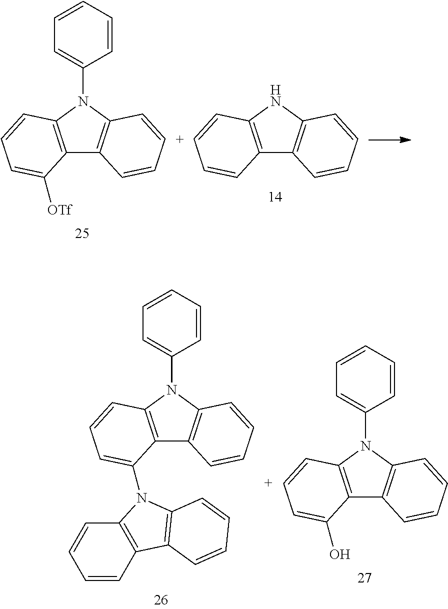 [SMILES: O=S(=O)(Oc1cccc2c1c1ccccc1n2-c1ccccc1)C(F)(F)F ; [C+]1=Cc2[nH]c3ccccc3c2C=C1 ; c1ccc(-n2c3ccccc3c3c(-n4c5ccccc5c5ccccc54)cccc32)cc1 ; OC1=C[C+]=Cc2c1c1ccccc1n2-c1ccccc1]

TABLE 11^{[a]}

| Ex. | Base | Y₂₅ [%] | Y₁₄ [%] | Y₂₆ [%] | Y₂₇ [%] |
|---|---|---|---|---|---|
| 1 | LiOtBu | 61.5 | 13.5 | 18.1 | 4.1 |
| 2 (comp.) | NaOtBu | 0 | 92.4 | 0 | 2.0 |

^{[a]}Reaction conditions: 0.50 mmol 9-phenyl-9H-carbazol-4-yl trifluoromethanesulfonate 25, 0.55 mmol carbazole 14, 220 mol % base, 2 mol % Pd(OAc)₂, 4 mol % XPhos, 5.7 ml toluene, 110° C., 16 h. Area percent determined by HPLC.

### Example 12

### Reaction of Electron-Rich Triflate With Sterically Demanding Secondary Amine

In this example, para-methoxyphenyl triflate as aryl compound is reacted with para-biphenyl-(2,4-diphenylphenyl)amine as a sterically demanding secondary amine (table 12). The reaction with LiOtBu as base leads to a good yield here, whereas almost no conversion to the tertiary amine is observed with NaOtBu as base.

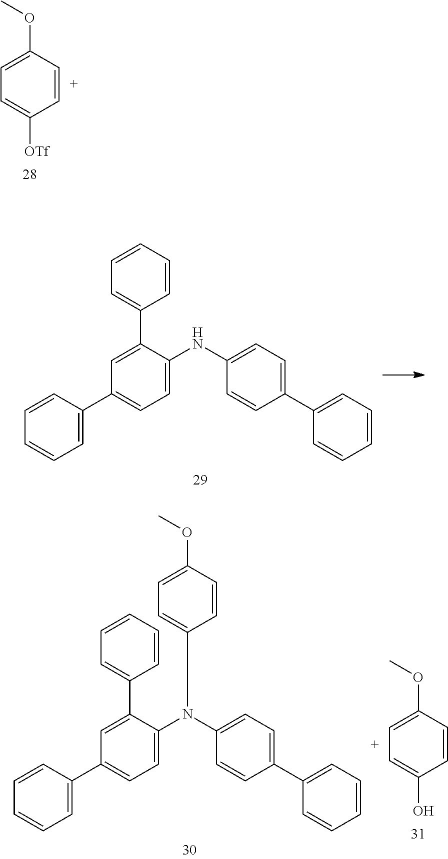

TABLE 12^{[a]}

| Ex. | Base | Y₂₈ [%] | Y₂₉ [%] | Y₃₀ [%] | Y₃₁ [%] |
|---|---|---|---|---|---|
| 1 | LiOtBu | 3.7 | 59.5 | 28.2 | k.A. |
| 2 (comp.) | NaOtBu | 0 | 95.3 | <1.0 | k.A. |

^{[a]}Reaction conditions: 0.76 mmol 4-methoxyphenyl trifluoromethanesulfonate 28, 0.83 mmol 2, 4-diphenyl-N-(4-phenylphenyl)aniline 29, 220 mol % base, 2 mol % Pd(OAc)₂, 4 mol % XPhos, 5.7 ml toluene, 110° C., 16 h. Area percent determined by HPLC.

### Example 13

### Selectivity of the Reaction of Chloroaryl Triflate With Sterically Demanding Secondary Amine

In this example, the aryl compound used is para-chlorophenyl triflate, which has two potential leaving groups in the form of the chlorine group and the triflate group. This is reacted selectively at the triflate group with ortho-biphenyl(phenyl)amine as a sterically demanding amine. The reaction with LiOtBu as base leads to a good yield here, whereas almost no conversion to the tertiary amine is observed with NaOtBu as base.

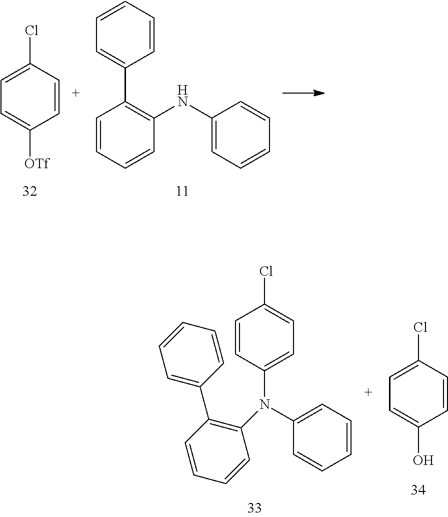

TABLE 13^{[a]}

| Ex. | Base | Y₃₂ [%] | Y₁₁ [%] | Y₃₃ [%] | Y₃₄ [%] |
|---|---|---|---|---|---|
| 1 | LiOtBu | 1.7 | 20.3 | 71.3 | — |
| 2 (comp.) | NaOtBu | 1.5 | 85.6 | 5 | — |

^{[a]}Reaction conditions: 0.76 mmol 4-chlorophenyl trifluoromethanesulfonate 32, 0.84 mmol biphenyl-2-yl(phenyl)amine 11, 220 mol % base, 2 mol % Pd(OAc)₂, 4 mol % XPhos, 5.7 ml toluene, 110° C., 15 h. Area percent determined by HPLC.

### Example 14

### Reaction of Phenyl Triflate With a Primary Amine to Give a Secondary Amine

In this example, phenyl triflate is reacted with aniline as primary arylamino compound (table 14). The reaction with LiOtBu as base leads to a distinctly better yield here compared to NaOtBu.

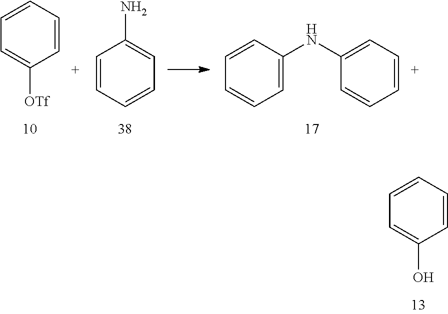

TABLE 14^{[a]}

| Ex. | Base | Y₁₀ [%] | Y₃₈ [%] | Y₁₇ [%] | Y₁₃ [%] |
|---|---|---|---|---|---|
| 1 | LiOtBu | 0 | 1.2 | 98.8 | — |
| 2 (comp.) | NaOtBu | 0 | 17.2 | 82.8 | — |

^{[a]}Reaction conditions: 0.88 mmol phenyl trifluoromethanesulfonate 10, 0.97 mmol aniline 38, 220 mol % base, 2 mol % Pd(OAc)₂, 4 mol % XPhos, 5.7 ml toluene, 110° C., 15 h. Area percent determined by HPLC.

### Example 15

### Reaction of Phenylsulfonate With Sterically Demanding Secondary Amine

In this example, a phenylsulfonate is used as leaving group. For this purpose, para-tolyl phenylsulfonate is reacted with ortho-biphenyl(phenyl)amine as a sterically demanding secondary amine (table 15). The reaction with LiOtBu as base leads to a distinctly better yield here compared to NaOtBu.

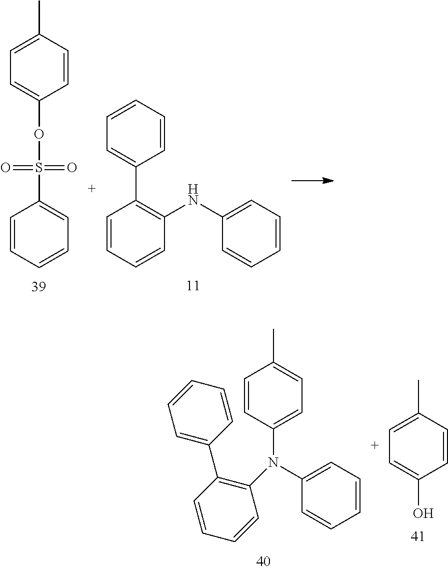

TABLE^{[a]}

| | | | Y₃₉ | Y₁₁ | Y₄₀ | Y₄₁ |
|---|---|---|---|---|---|---|
| Ex. | | Base | [%] | [%] | [%] | [%] |
| 1 | XPhos | LiOtBu | 7.4 | 52.4 | 32.7 | — |
| 2 | XPhos | NaOtBu | 3.12 | 81.3 | 10.9 | — |
| (comp.) | | | | | | |
| 3 | BrettPhos | LiOtBu | 3.6 | 68.3 | 23.6 | — |
| 4 | BrettPhos | NaOtBu | 0 | 92.4 | 4 | — |
| (comp.) | | | | | | |

^{[a]}Reaction conditions: 0.81 mmol phenyl 4-methylbenzenesulfonate 39,
0.88 mmol bipheny1-2-yl(phenyl)amine
11, 220 mol % base,
2 mol % Pd(OAc)₂,
4 mol % ligand,
5.7 ml toluene,
110° C., 15 h. Area percent determined by HPLC.

### Example 16

### Conversion on a Larger Scale

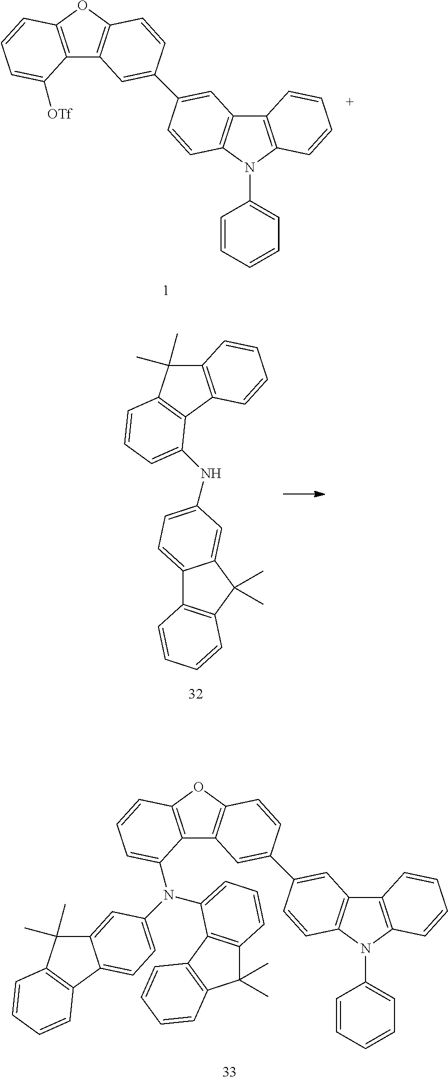 [SMILES: O=S(=O)(Oc1cccc2oc3ccc(-c4ccc5c(c4)c4ccccc4n5-c4ccccc4)cc3c12)C(F)(F)F ; CC1(C)c2ccccc2-c2ccc(Nc3cccc4c3-c3ccccc3C4(C)C)cc21 ; CC1(C)c2ccccc2-c2ccc(N(c3cccc4c3-c3ccccc3C4(C)C)c3cccc4oc5ccc(-c6ccc7c(c6)c6ccccc6n7-c6ccccc6)cc5c34)cc21]

In an autoclave, 98.0 g (172 mmol) of 8-(9-phenyl-9H-carbazol-3-yl)-dibenzofuran-1-yl trifluoromethanesulfonate 1, 76.1 g (189 mmol, 110 mol %) of (9,9-dimethyl-9H-fluoren-4-yl)(9,9-dimethyl-9H-fluoren-2-yl)amine 32 and 30.3 g (379 mmol, 220 mol %) of LiOtBu are weighed out and suspended in 1.90 l of anhydrous toluene. The mixture is degassed and flooded with nitrogen three times. In parallel, in a glovebox under an argon atmosphere, 0.78 g (3.49 mmol, 2.03 mol %) of Pd(OAc)₂ and 3.37 g (7.07 mmol, 4.11 mol %) of XPhos are weighed out, dissolved in 100 ml of anhydrous toluene and stirred for 1 h. Subsequently, the catalyst solution is added to the reactant solution against a nitrogen flow and the mixture is stirred at 110° C. for 16 h. After cooling to room temperature, a 3% N-acetylcysteine solution is added and the mixture is stirred for 30 min. After phase separation, the organic phase is filtered through basic alumina and then concentrated. A mixture of n-heptane and ethanol is added to the residue, and the crystallized solid is purified further by two hot extractions with n-heptane/toluene over basic alumina. Subsequently, the solid obtained is dissolved in THF and filtered through basic alumina. The filtrate is concentrated, and n-heptane is added. The solid obtained is finally sublimed under high vacuum. Yield: 85.2 g (105 mmol, 65.0%), purity >99.9% by HPLC.

### Example 17

### Bromine-Selective Suzuki Reaction

This example describes the preparation of the reactant for example 16 that has a triflate leaving group from a compound having both triflate and bromine as leaving groups. This involves selective conversion of the bromine group in the Suzuki coupling.

### General Procedure

The reactant and the boronic acid are weighed out together with the catalyst and the ligand and dissolved in the solvent. The mixture is saturated with argon for 5 min and then stirred at the specified temperature for 16 h. After cooling to room temperature, a sample is taken and analyzed by HPLC or GC-MS. The results of the catalyst screening are collated in table 16.

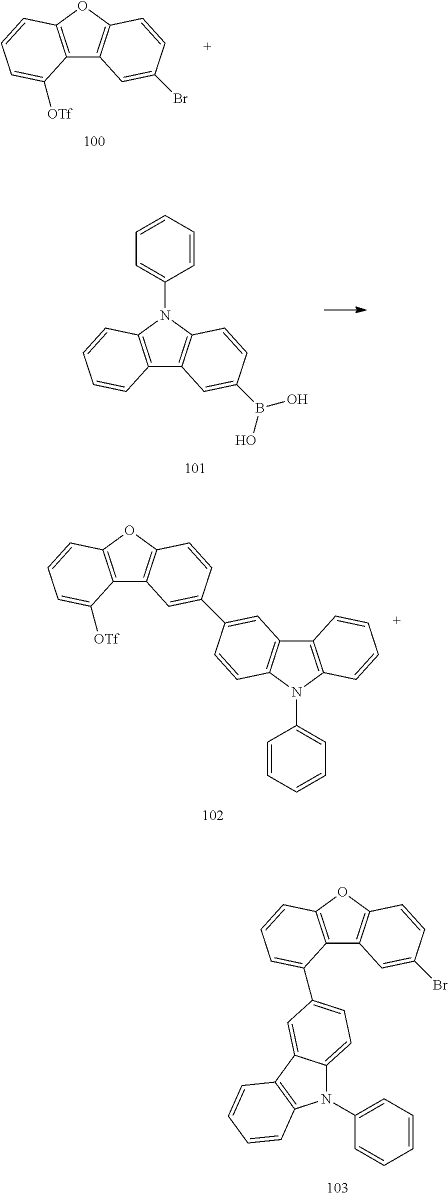 [SMILES: O=S(=O)(Oc1cccc2oc3ccc(Br)cc3c12)C(F)(F)F ; OB(O)c1ccc2c(c1)c1ccccc1n2-c1ccccc1 ; O=S(=O)(Oc1cccc2oc3ccc(-c4ccc5c(c4)c4ccccc4n5-c4ccccc4)cc3c12)C(F)(F)F ; Brc1ccc2oc3cccc(-c4ccc5c(c4)c4ccccc4n5-c4ccccc4)c3c2c1]

TABLE 16
Catalyst screening for the preparation of 102.^{[a]}

| Ex. | Catalyst system (ratio) | Y₁₀₀ [%] | Y₁₀₂ [%] | Y₁₀₃ [%] |
|---|---|---|---|---|
| 1 | Pd(PPh₃)₄ | 2 | 52 | 33 |
| 2 | Pd₂dba₃/PPh₃ (1:2) | 2 | 52 | 31 |
| 3 | Pd₂dba₃/P(o-Tol)₃ (1:2) | 0 | 80 | 3 |
| 4 | Pd₂dba₃/SPhos (1:2) | 5 | 11 | 73 |
| 5 | Pd₂dba₃/XPhos (1:2) | 4 | 24 | 61 |
| 6 | Pd₂dba₃/CMPhos (1:2) | 18 | 6 | 5 |
| 7 | Pd₂dba₃/PCy₃ (1:2) | 3 | 70 | 11 |
| 8 | Pd₂dba₃/P(t-Bu)₃ (1:2) | 3 | 7 | 1 |
| 9 | Pd₂dba₃/CataCxium A (1:2) | 0 | 74 | 9 |
| 10 | Pd-PEPPSI-iPr | 0 | 73 | 12 |

^{[a]}Reaction conditions: 1.27 mmol 8-bromodibenzofuran-1-yl trifluoromethanesulfonate 100, 1.52 mmol 9-phenylcarbazol-3-ylboronic acid, 2.53 mmol potassium carbonate, 1 mol % [Pd], 5.75 ml toluene, 2.00 ml water, 90° C., 16 h. Area percent determined by HPLC. Boronic acid content cannot be determined owina to co-elution with toluene.

The experimental parameters for the Pd₂dba₃/P(o-Tol)₃ catalyst/ligand system are optimized further (table 17).

TABLE 17
Optimization of the experimental parameters for the system
Pd₂dba₃/P(o-To1)₃ system for preparation of 102.^{[a]}

| | mol % | | | | | | |
|---|---|---|---|---|---|---|---|
| | Pd₂dba₃/ | K₂CO₃ | 101 | T | Y₁₀₀ | Y₁₀₂ | Y₁₀₃ |
| Ex. | P(o-To1)₃ | [mol %] | [mol %] | [° C.] | [%] | [%] | [%] |
| 1 | 0.25:1 | 200 | 120 | 80 | 0 | 78 | 0 |
| 2 | 0.25:1.5 | 200 | 120 | 80 | 0 | 79 | 0 |
| 3 | 0.5:2 | 200 | 110 | 80 | 0 | 83 | 0 |
| 4 | 0.5:2 | 120 | 110 | 80 | 0 | 83 | 0 |
| 5 | 0.5:2 | 120 | 100 | 70 | 2 | 83 | 0 |
| 6 | 0.5:2 | 120 | 80 | 70 | 5 | 79 | 0 |
| 7^{[b]} | 0.5:2 | 200 | 120 | 80 | 0 | 87 | 0 |
| | | | | | | (78^{[c]}) | |

^{[a]}Reaction conditions: 1.27 mmol 8-bromodibenzofuran-1-yl trifluoromethanesulfonate 100,
5.75 ml toluene,
2.00 ml water,
16 h. Area percent determined by HPLC. Boronic acid content could not be determined owing to co-elution with toluene.
^{[b]}Up-scaling reaction with 78 g 100.
^{[c]}Isolated yield.

Analogously to the reaction conditions in table 17, it is possible to prepare the following compounds:

| | | | Product |
|---|---|---|---|
| | | | content |
| Reactant 1 | Reactant 2 | Product | by GC-MS |
| 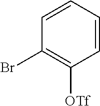 [SMILES: O=S(=O)(Oc1ccccc1Br)C(F)(F)F] | 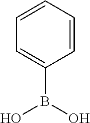 | 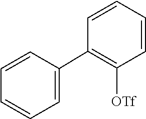 [SMILES: O=S(=O)(Oc1ccccc1-c1ccccc1)C(F)(F)F] | 81% |
| 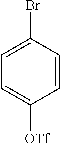 [SMILES: O=S(=O)(Oc1ccc(Br)cc1)C(F)(F)F] | 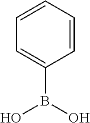 | 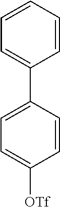 [SMILES: O=S(=O)(Oc1ccc(-c2ccccc2)cc1)C(F)(F)F] | 78% |
| 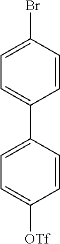 [SMILES: O=S(=O)(Oc1ccc(-c2ccc(Br)cc2)cc1)C(F)(F)F] | 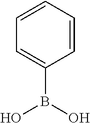 | 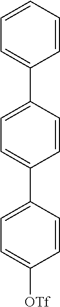 [SMILES: O=S(=O)(Oc1ccc(-c2ccc(-c3ccccc3)cc2)cc1)C(F)(F)F] | 95% |
| 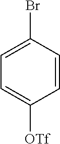 [SMILES: O=S(=O)(Oc1ccc(Br)cc1)C(F)(F)F] | 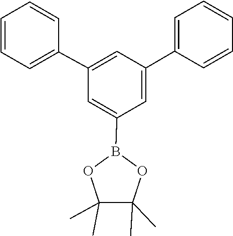 [SMILES: CC1(C)OB(c2cc(-c3ccccc3)cc(-c3ccccc3)c2)OC1(C)C] | 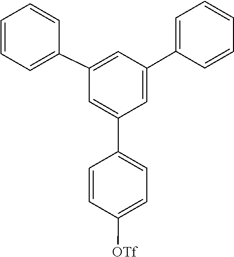 [SMILES: O=S(=O)(Oc1ccc(-c2cc(-c3ccccc3)cc(-c3ccccc3)c2)cc1)C(F)(F)F] | 90% |

### Example 18

### Triflate-Selective Suzuki Reaction

This example describes the preparation of reactants for the process of the invention that have a bromine leaving group from a compound having both triflate and bromine as leaving groups. This involves selective conversion of the triflate group in the Suzuki coupling.

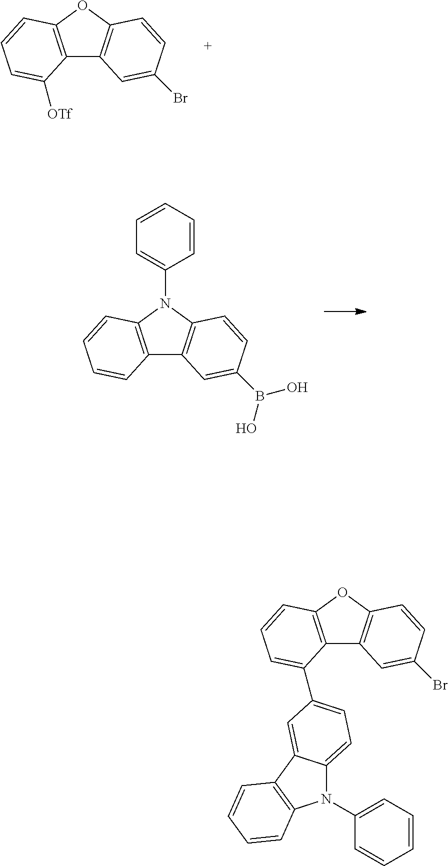 [SMILES: O=S(=O)(Oc1cccc2oc3ccc(Br)cc3c12)C(F)(F)F ; OB(O)c1ccc2c(c1)c1ccccc1n2-c1ccccc1 ; Brc1ccc2oc3cccc(-c4ccc5c(c4)c4ccccc4n5-c4ccccc4)c3c2c1]

20.0 g (49.2 mmol) of 8-bromodibenzofuran-1-yl trifluoromethanesulfonate, 14.8 g (51.6 mmol) of N-phenylcarbazole-3-boronic acid and 13.6 g (98.3 mmol) of potassium carbonate are suspended in 400 ml of toluene and 100 ml of water and inertized with argon for 20 min. After addition of 337 mg (0.37 mmol) of Pd₂dba₃ and 629 mg (1.47 mmol) of dppb, the mixture is heated to reflux for 48 h. After cooling to room temperature, the aqueous phase is separated off, and the organic phase is washed twice with water, dried over sodium sulfate and concentrated. The residue is crystallized from 500 ml of ethanol and is obtained in solid form. Yield: 22.0 g (45.1 mmol, 92%). Purity >98%.

1. A process for preparing a secondary or tertiary arylamino compound by a palladium-catalyzed coupling reaction between a primary or secondary arylamino compound and an aryl compound, characterized in that the process is conducted in the presence of lithium tert-butoxide (LiOtBu) as base.

2. The process as claimed in claim 1, characterized in that the arylamino compound used is a compound of formula (1) 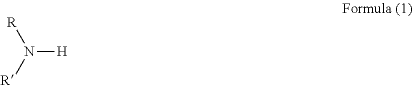
where the symbols used are as follows:
R is the same or different at each instance and is an aromatic or heteroaromatic ring system which has 5 to 60 aromatic ring atoms and may be substituted in each case by one or more R¹ radicals;
R′ is the same or different and is R or is H or D; at the same time, it is possible for R and R′ to be joined to one another directly or via an R¹ group and hence form a ring system together with the nitrogen atom to which they bind;
R¹ is the same or different at each instance and is selected from H, D, F, Cl, Br, I, B(OR²)₂, CHO, C(═O)R², CR²═C(R²)₂, CN, C(═O)OR², C(═O)N(R²)₂, Si(R²)₃, N(R²)₂, NO₂, P(═O)(R²)₂, OSO₂R², OR², S(═O)R², S(═O)₂R², a straight-chain alkyl, alkoxy or thioalkyl group having 1 to 20 carbon atoms or a branched or cyclic alkyl, alkoxy or thioalkyl group having 3 to 20 carbon atoms or an alkenyl or alkynyl group having 2 to 20 carbon atoms, where the abovementioned groups may each be substituted by one or more R² radicals and where one or more CH₂ groups in the abovementioned groups may be replaced by —R²C═CR²—, —C≡C—, Si(R²)₂, C═O, C═S, C═NR², —C(═O)O—, —C(═O)NR²—, NR², P(═O)(R²), —O—, —S—, SO or SO₂ and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, or an aryl- or heteroaryloxy group which has 5 to 30 aromatic ring atoms and may be substituted by one or more R² radicals; at the same time, it is possible for two or more R¹ radicals to be joined to one another and hence form a ring;
R² is the same or different at each instance and is selected from H, D, F, Cl, Br, I, B(OR³)₂, CHO, C(═O)R³, CR³═C(R³)₂, CN, C(═O)OR³, C(═O)N(R³)₂, Si(R³)₃, N(R³)₂, NO₂, P(═O)(R³)₂, OSO₂R³, OR³, S(═O)R³, S(═O)₂R³, a straight-chain alkyl, alkoxy or thioalkyl group having 1 to 20 carbon atoms or a branched or cyclic alkyl, alkoxy or thioalkyl group having 3 to 20 carbon atoms or an alkenyl or alkynyl group having 2 to 20 carbon atoms, where the abovementioned groups may each be substituted by one or more R³ radicals and where one or more CH₂ groups in the abovementioned groups may be replaced by —R³C═CR³—, —C≡C—, Si(R³)₂, C═O, C═S, C═NR³, —C(═O)O—, —C(═O)NR³—, NR³, P(═O)(R³), —O—, —S—, SO or SO₂ and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R³ radicals, or an aryl- or heteroaryloxy group which has 5 to 30 aromatic ring atoms and may be substituted by one or more R³ radicals; at the same time, it is possible for two or more R² radicals to be joined to one another and hence form a ring;
R³ is the same or different at each instance and is H, D, F or an aliphatic, aromatic or heteroaromatic organic radical having 1 to 20 carbon atoms, in which one or more hydrogen atoms may also be replaced by D or F; at the same time, two or more R³ substituents may be joined to one another and hence may form a ring.

3. The process as claimed in claim 2, characterized in that the aryl compound used is a compound of formula (2)
Ar—(X)ₙ Formula (2)
where R¹, R² and R³ have the definitions detailed in claim 2 and the further symbols and indices used are as follows:
Ar is an aromatic or heteroaromatic ring system which has 5 to 60 aromatic ring atoms and may be substituted by one or more R¹ radicals;
X is the same or different at each instance and is a leaving group;
n is an integer from 1 to 10.

4. The process as claimed in claim 2, characterized in that R and R′, when R′ is not H or D, are the same or different at each instance and are selected from the group consisting of phenyl, biphenyl, terphenyl, quaterphenyl, fluorenyl, spirobifluorenyl, naphthyl, indolyl, benzofuranyl, benzothienyl, carbazolyl, dibenzofuranyl, dibenzothienyl, indenocarbazolyl, indolocarbazolyl, phenanthryl and triphenylenyl, each of which may be substituted by one or more R¹ radicals.

5. The process as claimed in claim 3, characterized in that the aryl compound is substituted by a leaving group and the leaving group or the X group in the compound of the formula (2) is selected from the group consisting of optionally substituted alkylsulfonate, optionally substituted arylsulfonate, halide and diazonium.

6. The process as claimed in claim 5, characterized in that the aryl compound is substituted by a leaving group and the leaving group or the X group in the compound of the formula (2) is selected from the group consisting of triflate, phenylsulfonate and tosylate.

7. The process as claimed in claim 3, characterized in that the aromatic or heteroaromatic ring system in the aryl compound or the Ar group in the compound of the formula (2) is selected from the group consisting of phenyl, biphenyl, terphenyl, quaterphenyl, fluorenyl, spirobifluorenyl, naphthyl, indolyl, benzofuranyl, benzothienyl, carbazolyl, dibenzofuranyl, dibenzothienyl, indenocarbazolyl, indolocarbazolyl, phenanthenryl and triphenylenyl, each of which may be substituted by one or more R¹ radicals.

8. The process as claimed in claim 1, characterized in that lithium tert-butoxide is used in an amount of 0.5 to 10 equivalents, based on the molar amount of aryl compound used.

9. The process as claimed in claim 1, characterized in that the catalyst used is PdCl₂, Pd(OAc)₂, (CH₃CN)₂PdCl₂, bis(dibenzylideneacetone)dipalladium or tris(dibenzylideneacetone)dipalladium together with at least one ligand.

10. The process as claimed in claim 1, characterized in that the ligands used for the catalyst are phosphines, phosphites, amines, aminophosphines or N-heterocyclic carbenes.

11. The process as claimed in claim 10, characterized in that the phosphine is selected from the group consisting of dicyclohexylphosphino-2′,6′-dimethoxybiphenyl, dicyclohexylphosphino-2′,6′-diisopropoxybiphenyl, di-tert-butyl(2′,4′,6′-triisopropyl-3,6-dimethoxybiphenyl-2-yl)phosphine, dicyclohexyl(2′,4′,6′-triisopropyl-3,6-dimethoxybiphenyl-2-yl)phosphine, trimethylphosphine, triethylphosphine, tripropylphosphine, triisopropylphosphine, tributylphosphine, tri-tert-butylphosphine, tricyclohexylphosphine, triphenylphosphine, di-tert-butylchlorophosphine, triphenylphosphine, tri(o-tolyl)phosphine, triisopropylphosphine, tricyclohexylphosphine, 2,2′-bis(diphenylphosphino)-1,1′-binaphthyl (BINAP), 1,2-bis(dimethylphosphino)ethane, 1,2-bis(diethylphosphino)ethane, 1,2-bis(dipropylphosphino)ethane, 1,2-bis(diisopropylphosphino)ethane, 1,2-bis(dibutylphosphino)ethane, 1,2-bis(dicyclohexylphosphino)ethane, 1,3-bis(dicyclohexylphosphino)propane, 1,3-bis(diisopropylphosphino)propane, 1,4-bis(diisopropylphosphino)butane, 2,4-bis(dicyclohexylphosphino)pentane, 1,1′-bis(diphenylphosphino)ferrocene, SPhos, PCy₃, Cy-JohnPhos, CataCxium Pcy, APhos, XantPhos, dppf, XPhos and BrettPhos.

12. The process as claimed in claim 9, characterized in that the palladium compound and the phosphine ligand in the case of monophosphines are used in a Pd:phosphine ratio of 1:1 to 1:4, and in that the palladium compound and the phosphine ligand in the case of biphosphines are used in a Pd:phosphine ratio of 1:0.5 to 1:2.

13. The process as claimed in claim 1, characterized in that the process is conducted in one or more aprotic organic solvents.

14. The process as claimed in claim 1, characterized in that the process is conducted under inert gas atmosphere.

15. The use of LiOtBu as base in a palladium-catalyzed coupling reaction between an arylamino compound and an aryl compound.

16. The process as claimed in claim 1, characterized in that the process is conducted in one or more aprotic organic solvents selected from the group consisting of benzene, toluene, 1,2-xylene, 1,3-xylene, 1,4-xylene, mesitylene, tetrahydrofuran (THF), 1,4-dioxane, dimethoxyethane (dme) and bis(2-methoxyethyl) ether (diglyme).
